SEQUENCE LISTING (11 sequences)

SEQ ID NO: 1 - DNA, 20 bp, Artificial Sequence
  unknown
tccatgacgttcctgacgtt

SEQ ID NO: 2 - DNA, 18 bp, Artificial Sequence
  unknown
tctcccagcgtgcgccat

SEQ ID NO: 3 - DNA, 30 bp, Artificial Sequence
  unknown
accgatgacgtcgccggtgacggcaccacg

SEQ ID NO: 4 - DNA, 24 bp, Artificial Sequence
  unknown
tcgtcgttttgtcgttttgtcgtt

SEQ ID NO: 5 - DNA, 20 bp, Artificial Sequence
  unknown
tccatgacgttcctgatgct

SEQ ID NO: 6 - DNA, 22 bp, Artificial Sequence
  unknown
tcgacgttttcggcgcgcgccg

SEQ ID NO: 7 - DNA, 378 bp, Artificial Sequence
  LTB,LTB-cys coding sequence
atgaataaagtaaaatgttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaactgctaa

SEQ ID NO: 8 - DNA, 429 bp, Artificial Sequence
  unknown
atgaataaagtaaaatgttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaacagccagcttgagagtataatcaactttgaaaaactgactgaatggcgcggccgctag

SEQ ID NO: 9 - DNA, 849 bp, Artificial Sequence
  unknown
atgggccaccatcaccatcaccattcttctggtgcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagagttcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccggatcccagcttgagagtataatcaactttgaaaaactgactgaatggtga

SEQ ID NO: 10 - DNA, 1200 bp, Artificial Sequence
  unknown
atgggccaccatcaccatcaccattcttctggtgcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagagttcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccggatccgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatga

SEQ ID NO: 11 - DNA, 1854 bp, Artificial Sequence
  unknown
atggccgaggaagccttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacctgcactgccagcttgagagtataatcaactttgaaaaactgactgaatggtgcatgcagggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctgaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtga

(19) United States
(12) Patent Application Publication
(10) Pub. No.: US 2009/0035330 A1
(43) Pub. Date: Feb. 5, 2009

(54) Vaccine Composition Comprising B-Subunit Of E. coli Heat Toxin And An Antigen And An Adjuvant

(76) Inventor: Marianne Dewerchin, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/913,981

(22) PCT Filed: May 18, 2006
(86) PCT No.: PCT/GB2006/001832

§ 371 Date: Nov. 9, 2007

(30) Foreign Application Priority Data
May 19, 2005 (GB) 0510280.1
Nov. 30, 2005 (GB) 05244707.4

(51) Int. Cl. A61K 39/108

(52) U.S. Cl. 424/241.1

(57) ABSTRACT

The present invention provides a vaccine composition comprising the B-subunit of E. coli heat labile toxin or a derivative thereof with equal or greater than 90% homology complexed with an antigen and an adjuvant.

The present invention provides improved vaccine compositions, methods for making them and their use in medicine. In particular the present invention provides adjuvanted vaccine compositions which comprise an agent which can improve MHC class I presentation of an antigen, and an antigen formulated with an adjuvant.

The development of vaccines which require a predominant induction of a cellular response remains a challenge. Because CD8+ T cells, the main effector cells of the cellular immune response, recognise antigens that are synthesized in pathogen-infected cells, successful vaccination requires the synthesis of immunogenic antigens in cells of the vaccinee. This can be achieved with live-attenuated vaccines, however they also present significant limitations. First, there is a risk of infection, either when vaccinees are immunosuppressed, or when the pathogen itself can induce immunosuppression (e.g. Human Immunodeficiency Virus). Second, some pathogens are difficult or impossible to grow in cell culture (e.g. Hepatitis C Virus). Other existing vaccines such as inactivated whole-cell vaccines or alum adjuvanted, recombinant protein subunit vaccines are notably poor inducers of CD8 responses.

For these reasons, alternative approaches are being developed: live vectored vaccines, plasmid DNA vaccines, synthetic peptides or specific adjuvants. Live vectored vaccines are good at inducing a strong cellular response but pre-existing (e.g. adenovirus) or vaccine-induced immunity against the vector may jeopardize the efficiency of additional vaccine dose (Casimiro et al, JOURNAL OF VIROLOGY, June 2003, p. 6305-6313). Plasmid DNA vaccines also can induce a cellular response (Casimiro et al, JOURNAL OF VIROLOGY, June 2003, p. 6305-6313) but it remains weak in humans (Mc Conkey et al, Nature Medicine 9, 729-735, 2003) and the antibody response is very poor. In addition, synthetic peptides are currently being evaluated in clinical trials (Khong et al, J Immunother 2004;27:472-477), but the efficacy of such vaccines encoding a limited number of T cell epitopes may be hampered by the appearance of vaccine escape mutants or by the necessity of first selecting for HLA-matched patients.

Alternative approaches aimed at improving MHC class I presentation have also been described, based on antigen delivery using non-live vectors. Some non-live vectors are derived from bacterial toxins, for example Anthrax LFn toxin (Ballard et al (1996) PNAS USA 93 pp12531-12534), B. pertussis adenylate cyclase toxin (Fayolle et al (1996) J. Immunology 156 p 4697-4706), Pseudomonas Exotoxin A (Donnelly et al PNAS USA (1993) 90 pp 3530-3534)., or E. coli Heat-Labile toxin (Partidos et al. Immunology (1996) 89 pp 483-487)

The limitations of vaccine antigens and delivery systems justify the search for new vaccine compositions. The present inventors have found that the inclusion of adjuvants in compositions comprising non-live vectors derived from bacterial toxins can have a beneficial effect on the resulting immune response, in particular CD8 specific responses. It is thought that this beneficial effect is due to the combination of the activation of the immune response given by the adjuvant with the correct delivery of an antigen provided by the agent which targets the MHC1 pathway rather than to an additional adjuvant effect provided by such agent. Previous studies using vaccine compositions containing LT B-subunit and an antigen administered with an adjuvant showed no synergistic effect to the strength of the immune response (McCluskie et al. 2000 Mol Medicine 6 pp 867-877; McCluskie et al (2001) Vaccine 19 pp 3759-3768).

Therefore the present invention provides a vaccine composition comprising the B-subunit of E. coli heat labile toxin or a derivative thereof with equal or greater than 90% homology complexed with an antigen and further comprising an adjuvant.

In a further embodiment the B-subunit of E. coli heat labile toxin or a derivative with equal or greater than 90% homology is able to bind the G_{M1} receptor. In a further embodiment B-subunit of E. coli heat labile toxin or a derivative with equal or greater than 90% homology is able to target an antigen into the MHC class I pathway as measured by the methods described in section 2.1

The term “vaccine composition” used herein is defined as composition used to elicit an immune response against an antigen within the composition in order to protect or treat an organism against disease.

In the context of the invention, the word toxin is intended to mean toxins that have been detoxified such that they are no longer toxic to humans, or a toxin subunit or fragment thereof that are substantially devoid of toxic activity in humans.

The preferred non-live vector based on detoxified toxins is the B subunit from E. coli labile toxin (LT). In the preferred embodiment, the non-live vector is the B subunit from E. coli labile toxin type I (LTI).

Further non-live vectors based on detoxified toxins include the amino terminal domain of the anthrax lethal factor (LF), P. aeruginosa exotoxin A, and the adenylate cyclase A from B. pertussis. IFor example, the non-live vector is derived from a toxin which is a family of the AB5 family, for example, the cholera toxin (CT), the Bordatella Pertussis toxin (PT) as well as the recently identified subtilase cytotoxins. (Paton et al, J Exp Med 2004, Vol 200 pp 35-46).

The labile toxin (LT) of E. coli consists of two subunits, a pentameric B subunit and a monomeric A subunit. The A subunit is responsible for toxicity, whilst the B subunit is responsible for transport into the cell. LT binds the G_{M1} ganglioside receptor.

A derivative of E. coli heat-labile toxin with equal or greater the 90% homology has greater than 90% homology at the amino acid level. In another embodiment the protein has equal or greater than 95% homology, for example 96, 97, 98 or 99%. For example, amino acid deletions may be made that do not affect function. In a further embodiment, a derivative is still able to bind the G_{M1} ganglioside receptor. In a further embodiment a derivative is still able to elicit an immune response against a complexed antigen as measured by the methods described in section 2.1. Whether a vector or equivalent binds the G_{M1} receptor may be determined, for example, by following the protocol set out in example 1.4 below.

The amino-terminal domain from B. Anthracis (anthrax) LF is known as LFn. It is the N-terminal 255 amino acids of LF. LF has been found to contain the information necessary for binding to protective antigen (PA) and mediating translocation. The domain alone lacks lethal potential, that depends on the putatively enzymatic carboxyl-terminal moiety (Arora and Leppla (1993) J. Biol Chem 268 pp 3334-3341). In addition, it was recently found that a fusion protein of the LFn domain with a foreign antigen can induce CD8 T cell immune responses even in the absence of PA (Kushner et al (2003), PNAS 100 pp 6652-6657) suggesting that LFn may be used without PA as a carrier to deliver antigens into the cytosol.

Donnelly et al (Supra) demonstrate that the toxic domain may be removed from P. aeroginosa and the remainder of the toxin may still mediate transport of an antigen into the cell. In addition, deletion of aa from the full-length toxin does not impairs its ability to access to the cytosol but renders it nontoxic since this mutation eliminates the ADP-ribosylating activity. Based on this mutant, chimeras can be constructed that encode antigenic sequences of various sizes (Fitzgerald, J Biol Chem, Vol. 273, Issue 16, 9951-9958, Apr. 17, 1998.

The adenylate cyclase toxin binds the CD11b receptor at the surface of dendritic cells. Recombinant toxoids bearing CD8⁺ T-cell epitopes are able to induce specific CTL responses in mice and protection against experimental tumours has been demonstrated (Fayolle et al, J Immunol 1999, 162 pp 4157-4162). Surface presentation of the delivered epitopes occurs via the classical MHC class I pathway

Other vectors may be derived by using a receptor or receptor mimic that a bacterial toxin is known to bind to for screening a phage-display library. Such a technique would provide peptides (for example up to 20 amino acids or so in length) that could bind the same receptor as the bacterial toxin, but would have little or no sequence similarity to the toxin. This technique has been shown to be an effective way of generating peptides that bind to the GB3 receptor (Miura et al Biochimica et Biphysica Acta 1673 (2004) pp 131-138) and the GM1 receptor (Matsubara et al FEBS letters 456 (1999) 253-256. It is likely that such peptides could act as vectors in the same way as the bacterial toxins which bind to the same receptors. Such peptides are considered to fall within the definition “vector derived from a bacterial toxin” as they are derived by screening at the same receptor as that that the bacterial toxin binds to. In one embodiment, however, the vector of the invention that is “derived from a bacterial toxin” is actually a bacterial toxin or an immunologically functional equivalent thereof.

Not included within the scope of the present invention are those non-live vectors or immunologically functional equivalents thereof which are able to bind the Gb3 receptor. Whether a vector or equivalent binds the Gb3 receptor may be determined, for example, by following the protocol set out in section 1.5 below.

The compositions of the invention are capable of improving a CD8 specific immune response to the antigen complexed to a protein of the invention. Improvement is measured by looking at the response to a composition of the invention comprising a antigen complexed to a protein of the invention and comprising an adjuvant when compared to the response to a composition comprising a antigen complexed to a protein of the invention with no adjuvant, or the response to a formulation comprising the antigen with adjuvant. Improvement may be defined as an increase in the level of the immune response, the generation of an equivalent immune response with a lower dose of antigen, an increase in the quality of the immune response, an increase in the persistency of the immune response, or any combination of the above. Such an improvement may be seen following a first immunization, and/or may be seen following subsequent immunizations.

Particular adjuvants are those selected from the group of metal Salts, oil in water emulsions, Toll like receptors ligands, (in particular Toll like receptor 2 ligand, Toll like receptor 3 ligand, Toll like receptor 4 ligand, Toll like receptor 7 ligand, Toll like receptor 8 ligand and Toll like receptor 9 ligand), saponins or combinations thereof. In one embodiment, the toll like receptor ligand is a receptor agonist. In another embodiment, the toll like receptor ligand is a receptor antagonist. The term “ligand” as used throughout the specification and the claims is intended to mean an entity that can bind to the receptor and have an effect, either to upregulate or downregulate the activity of the receptor.

The adjuvant is preferably selected from the group: a saponin, lipid A or a derivative thereof, an immunostimulatory oligonucleotide, an alkyl glucosaminide phosphate, or combinations thereof. A further preferred adjuvant is a metal salt in combination with another adjuvant. It is preferred that the adjuvant is a Toll like receptor ligand in particular an ligand of a Toll like receptor 2, 3, 4, 7, 8 or 9, or a saponin, in particular Qs21. It is further preferred that the adjuvant system comprises two or more adjuvants from the above list. In particular the combinations preferably contain a saponin (in particular Qs21) adjuvant and/or a Toll like receptor 9 ligand such as a immunostimulatory oligonucleotide containing CpG or other immunostimulatory motifs such as CpR where R is a non-natural guanosine nucleotide. Other preferred combinations comprise a saponin (in particular QS21) and a Toll like receptor 4 ligand such as monophosphoryl lipid A or its 3 deacylated derivative, 3 D-MPL, or a saponin (in particular QS21) and a Toll like receptor 4 ligand such as an alkyl glucosaminide phosphate. Other preferred combinations comprise a TLR 3 or 4 ligand in combination with a TLR 8 or 9 ligand.

Particularly preferred adjuvants are combinations of 3D-MPL and QS21 (EP 0 671 948 B1), oil in water emulsions comprising 3D-MPL and QS21 (WO 95/17210, WO 98/56414), or 3D-MPL formulated with other carriers (EP 0 689 454 B1). Other preferred adjuvant systems comprise a combination of 3 D MPL, QS21 and a CpG oligonucleotide as described in U.S. Pat. No. 6,558,670, U.S. Pat. No. 6,544,518.

In an embodiment the adjuvant is a Toll like receptor (TLR) 4 ligand, preferably an ligand such as a lipid A derivative particularly monophosphoryl lipid A or more particularly 3 Deacylated monophoshoryl lipid A (3 D-MPL).

3 D-MPL is sold under the trademark MPL® by GSK biologicals and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR ligands including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S, 9 R)-3-[(R)-dodecanoyloxytetradecanoylamino]-4-oxo-5-aza-9(R)-[(R)-3-hydroxytetradecanoylamino]decan-1,10-diol, 1,10-bis(dihydrogenophosphate) (WO99/64301 and WO 00/0462)

OM 197 MP-Ac DP (3S-, 9R)-3-[(R)-dodecanoyloxytetradecanoylamino]-4-oxo-5-aza-9-[(R)-3-hydroxytetradecanoylamino]decan-1,10-diol, 1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Another prefered immunostimulant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p 243-254). Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly preferred, these formulations further comprise a sterol (WO96/33739). The saponins forming part of the present invention may be separate in the form of micelles, mixed micelles (preferentially, but not exclusively with bile salts) or may be in the form of ISCOM matrices (EP 0 109 942 B1), liposomes or related colloidal structures such as worm-like or ring-like multimeric complexes or lipidic/layered structures and lamellae when formulated with cholesterol and lipid, or in the form of an oil in water emulsion (for example as in WO 95/17210). The saponins may preferably be associated with a metallic salt, such as aluminium hydroxide or aluminium phosphate (WO 98/15287). Preferably, the saponin is presented in the form of a liposome, ISCOM or an oil in water emulsion.

Immunostimulatory oligonucleotides or any other Toll-like receptor (TLR) 9 ligand may also be used. The preferred oligonucleotides for use in adjuvants or vaccines of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

OLIGO 1(SEQ ID NO:1):
TCC ATG ACG TTC CTG ACG TT (CpG 1826)
OLIGO 2(SEQ ID NO:2):
TCT CCC AGC GTG CGC CAT (CpG 1758)
OLIGO 3(SEQ ID NO:3):
ACC GAT GAC GTC GCC GGT GAC GGC ACC ACG
OLIGO 4(SEQ ID NO:4):
TCG TCG TTT TGT CGT TTT GTC GTT (CpG 2006)
OLIGO 5(SEQ ID NO:5):
TCC ATG ACG TTC CTG ATG CT (CpG 1668)
OLIGO 6(SEQ ID NO:6):
TCG ACG TTT TCG GCG CGC GCC G (CpG 5456)

Alternative CpG oligonucleotides may comprise the preferred sequences above in that they have inconsequential deletions or additions thereto.

Alternative immunostimulatory oligonucleotides may comprise modifications to the nucleotides. For example, WO0226757 and WO03507822 disclose modifications to the C and G portion of a CpG containing immunostimulatory oligonucleotides.

The immunostimulatory oligonucleotides utilised in the present invention may be synthesized by any method known in the art (for example see EP 468520). Conveniently, such oligonucleotides may be synthesized utilising an automated synthesizer.

Examples of a TLR 2 ligand include peptidoglycan or lipoprotein. Imidazoquinolines, such as Imiquimod and Resiquimod are known TLR7 ligands. Single stranded RNA is also a known TLR ligand (TLR8 in humans and TLR7 in mice), whereas double stranded RNA and poly IC (polyinosinic-polycytidylic acid—a commercial synthetic mimetic of viral RNA). are exemplary of TLR 3 ligands. 3D-MPL is an example of a TLR4 ligand whilst CPG is an example of a TLR9 ligand

The non-live vector derived from a bacterial toxin or immunologically functional equivalent thereof and the antigen are complexed together. By complexed is meant that the non-live vector derived from a bacterial toxin or immunologically functional equivalent thereof and the antigen are physically associated, for example via an electrostatic or hydrophobic interaction or a covalent linkage. In a preferred embodiment the non-live vector derived from a bacterial toxin or immunologically functional equivalent thereof are covalently linked either as a fusion protein or chemically coupled, for example via a cysteine residue. In embodiments of the invention more than one antigen is linked to each non-live vector or immunologically functional equivalent thereof such as 2,3,4,5 6 antigen molecules per vector. When more than one antigen is present, these antigens may all be the same, one or more may be different to the others, or all the antigens may be different to each other.

The antigen itself may be a peptide, or a protein encompassing one or more epitopes of interest. It is a preferred embodiment that the antigen is selected such that when formulated in the manner contemplated by the invention it provides immunity against intracellular pathogens such as HIV, tuberculosis, Chlamydia, HBV, HCV, and Influenza The present Invention also finds utility with antigens which can raise relevant immune responses against benign and proliferative disorders such as Cancers.

Preferably the vaccine formulations of the present invention contain an antigen or antigenic composition capable of eliciting an immune response against a human pathogen, which antigen or antigenic composition is derived from HIV-1, (such as gag or fragments thereof, such as p24, tat, nef, envelope such as gp120 or gp160, or fragments of any of these), human herpes viruses, such as gD or derivatives thereof or Immediate Early protein such as ICP27 from HSV1 or HSV2, cytomegalovirus ((esp Human)(such as gB or derivatives thereof), Rotaviral antigen, Epstein Barr virus (such as gp350 or derivatives thereof), Varicella Zoster Virus (such as gpI, II and IE63), or from a hepatitis virus such as hepatitis B virus (for example Hepatitis B Surface antigen or a derivative thereof), or antigens from hepatitis A virus, hepatitis C virus and hepatitis E virus, or from other viral pathogens, such as paramyxoviruses: Respiratory Syncytial virus (such as F G and N proteins or derivatives thereof), parainfluenza virus, measles virus, mumps virus, human papilloma viruses (for example HPV 6, 11, 16, 18,) flaviviruses (e.g. Yellow Fever Virus, Dengue Virus, Tick-borne encephalitis virus, Japanese Encephalitis Virus) or Influenza virus purified or recombinant proteins thereof, such as HA, NP, NA, or M proteins, or combinations thereof), or derived from bacterial pathogens such as Neisseria spp, including N. gonorrhea and N. meningitidis (for example, transferrin-binding proteins, lactoferrin binding proteins, PilC, adhesins); S. pyogenes (for example M proteins or fragments thereof, C5A protease,), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd 14-DPV-MTI-MSL, DPV-MTI-MSL-mTCC2, TbH9-DPV-MTI (WO 99/51748).

Most preferred antigens for Chlamydia include for example the High Molecular Weight Protein (HMW) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (for example, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B, non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof.

Derivatives of Hepatitis B Surface antigen are well known in the art and include, inter alia, those PreS1, PreS2 S antigens set forth described in European Patent applications EP-A-414 374; EP-A-0304 578, and EP 198-474. In one preferred aspect the vaccine formulation of the invention comprises the HIV-1 antigen, gp120, especially when expressed in CHO cells. In a further embodiment, the vaccine formulation of the invention comprises gD2t as hereinabove defined.

In a preferred embodiment of the present invention the vaccine compositions comprise antigen derived from the Human Papilloma Virus (HPV) considered to be responsible for genital warts (HPV 6 or HPV 11 and others), and the HPV viruses responsible for cervical cancer (HPV16, HPV18 and others).

Particularly preferred forms of genital wart prophylactic, or therapeutic, vaccine comprise L1 protein, and fusion proteins comprising one or more antigens selected from the HPV proteins E1, E2, E5, E6, E7, L1, and L2.

The most preferred forms of fusion protein are: L2E7 as disclosed in WO 96/26277, and proteinD(1/3)-E7 disclosed in WO99/10375.

A preferred HPV cervical infection or cancer, prophylaxis or therapeutic vaccine composition may comprise HPV 16 or 18 antigens.

Particularly preferred HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277).

Alternatively the HPV 16 or 18 early proteins E6 and E7, may be presented in a single molecule, preferably a Protein D-E6/E7 fusion. Such vaccine may optionally contain either or both E6 and E7 proteins from HPV 18, preferably in the form of a Protein D-E6 or Protein D-E7 fusion protein or Protein D E6/E7 fusion protein.

The vaccine of the present invention may additionally comprise antigens from other HPV strains, preferably from strains HPV 31 or 33.

Vaccine compositions of the present invention further comprise antigens derived from parasites that cause Malaria, for example, antigens from Plasmodia falciparum including circumsporozoite protein (CS protein), RTS,S, MSP1, MSP3, LSA1, LSA3, AMA1 and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in International Patent Application No. PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in International Patent Application No. PCT/GB89/00895, published under WO 90/01496. Plasmodia antigens that are likely candidates to be components of a multistage Malaria vaccine are P. falciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp. One embodiment of the present invention is a malaria vaccine wherein the antigen preparation comprises RTS,S or CS protein or a fragment thereof such as the CS portion of RTS,S, in combination with one or more further malarial antigens, either or both of which may be attached to the Shiga toxin B subunit in accordance with the invention. The one or more further malarial antigens may be selected for example from the group consisting of MPS1, MSP3, AMA1, LSA1 or LSA3.

The formulations may also contain an anti-tumour antigen and be useful for the immunotherapeutic treatment of cancers. For example, the adjuvant formulation finds utility with tumour rejection antigens such as those for prostrate, breast, colorectal, lung, pancreatic, renal or melanoma cancers. Exemplary antigens include MAGE 1 and MAGE 3 or other MAGE antigens (for the treatment of melanoma), PRAME, BAGE, or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p 293. Indeed these antigens are expressed in a wide range of tumour types such as melanoma, lung carcinoma, sarcoma and bladder carcinoma. Other tumour-specific antigens are suitable for use with the adjuvants of the present invention and include, but are not restricted to tumour-specific gangliosides, Prostate specific antigen (PSA) or Her-2/neu, KSA (GA733), PAP, mammaglobin, MUC-1, carcinoembryonic antigen (CEA) or p501S (prostein). Accordingly in one aspect of the present invention there is provided a vaccine comprising an adjuvant composition according to the invention and a tumour rejection antigen.

It is a particularly preferred aspect of the present invention that the vaccines comprise a tumour antigen such as prostrate, breast, colorectal, lung, pancreatic, renal, ovarian or melanoma cancers. Accordingly, the formulations may contain tumour-associated antigen, as well as antigens associated with tumour-support mechanisms (e.g. angiogenesis, tumour invasion). Additionally, antigens particularly relevant for vaccines in the therapy of cancer also comprise Prostate-specific membrane antigen (PSMA), Prostate Stem Cell Antigen (PSCA), tyrosinase, survivin, NY-ESO1, prostase, PS108 (WO 98/50567), p501S (prostein), RAGE, LAGE, HAGE. Additionally said antigen may be a self peptide hormone such as whole length Gonadotrophin hormone releasing hormone (GnRH, WO 95/20600), a short 10 amino acid long peptide, useful in the treatment of many cancers, or in immunocastration.

Vaccines of the present invention may be used for the prophylaxis or therapy of allergy. Such vaccines would comprise allergen specific antigens, for example Der p1

The amount of antigen in each vaccine dose is selected as an amount which induces an immunoprotective response without significant, adverse side effects in typical vaccinees. Such amount will vary depending upon which specific immunogen is employed and how it is presented.

Generally, it is expected that each human dose will comprise 0.1-1000 μg of antigen, preferably 0.1-500 μg, preferably 0.1-100 μg, most preferably 0.1 to 50 μg. An optimal amount for a particular vaccine can be ascertained by standard studies involving observation of appropriate immune responses in vaccinated subjects. Following an initial vaccination, subjects may receive one or several booster immunisation adequately spaced. Such a vaccine formulation may be applied to a mucosal surface of a mammal in either a priming or boosting vaccination regime; or alternatively be administered systemically, for example via the transdermal, subcutaneous or intramuscular routes. Intramuscular administration is preferred.

The amount of 3 D MPL used is generally small, but depending on the vaccine formulation may be in the region of 1-1000 μg per dose, preferably 1-500 μg per dose, and more preferably between 1 to 100 μg per dose.

The amount of CpG or immunostimulatory oligonucleotides in the adjuvants or vaccines of the present invention is generally small, but depending on the vaccine formulation may be in the region of 1-1000 μg per dose, preferably 1-500 μg per dose, and more preferably between 1 to 100 μg per dose.

The amount of saponin for use in the compositions of the present invention may be in the region of 1-1000 μg per dose, preferably 1-500 μg per dose, more preferably 1-250 μg per dose, and most preferably between 1 to 100 μg per dose.

The formulations of the present invention maybe used for both prophylactic and therapeutic purposes. Accordingly the invention provides a vaccine composition as described herein for use in medicine.

In a further embodiment there is provided a method of treatment of an individual susceptible to or suffering from a disease by the administration of a composition as substantially described herein.

Also provided is a method to prevent an individual from contracting a disease selected from the group comprising infectious bacterial and viral diseases, parasitic diseases, particularly intracellular pathogenic disease, proliferative diseases such as prostate, breast, colorectal, lung, pancreatic, renal, ovarian or melanoma cancers; non-cancer chronic disorders, allergy comprising the administration of a composition as substantially described herein to said individual.

Furthermore, there is described a method of inducing a CD8+antigen specific immune response in a mammal, comprising administering to said mammal a composition of the invention. Further there is provided a method of manufacture of a vaccine comprising admixing an antigen in combination with a non-live vector or immunological functional equivalent thereof with an adjuvant.

Examples of suitable pharmaceutically acceptable excipients for use in the combinations of the present invention include, among others, water, phosphate buffered saline, isotonic buffer solutions

The present invention is exemplified by reference to the following examples and Figures. In all figures, adeno-ova (adenovirus vector containing OVA protein) was used as a positive control in first injection. P/B (prime/boost) is a positive control with first injection of Adeno-Ova, and second, boost injection of Ova in AS A.

The Following Figures Show the Effect of Adjuvant System A on the Immune Reponse to LT-Ova and LTcys-Ova

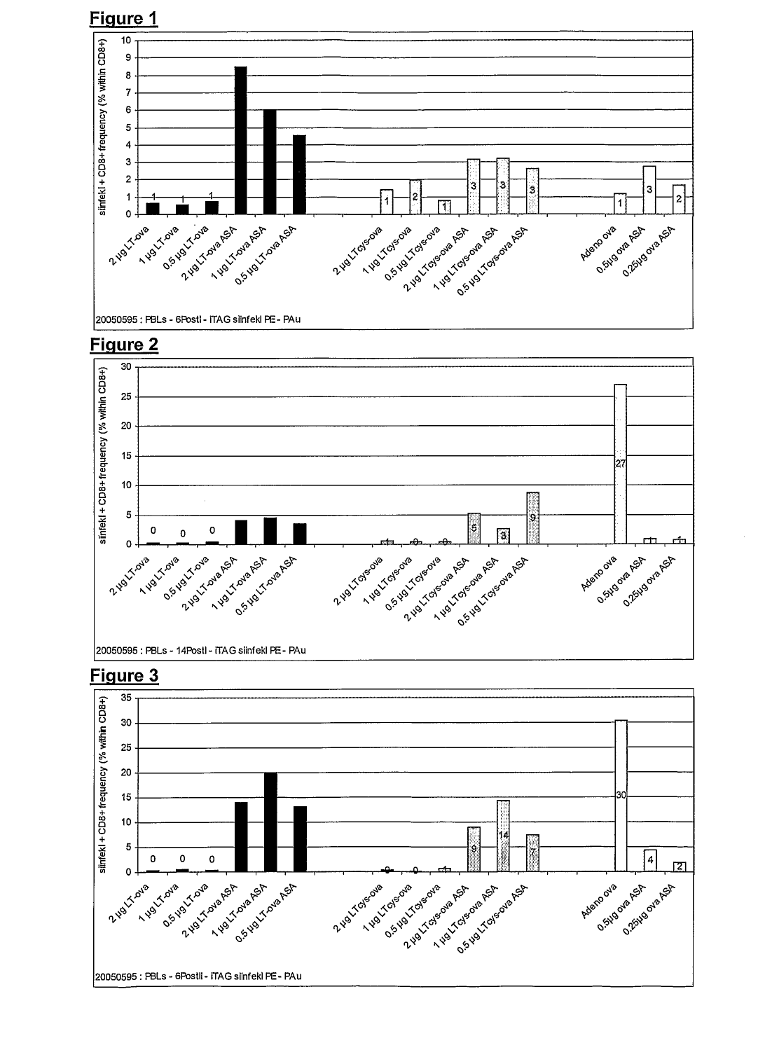

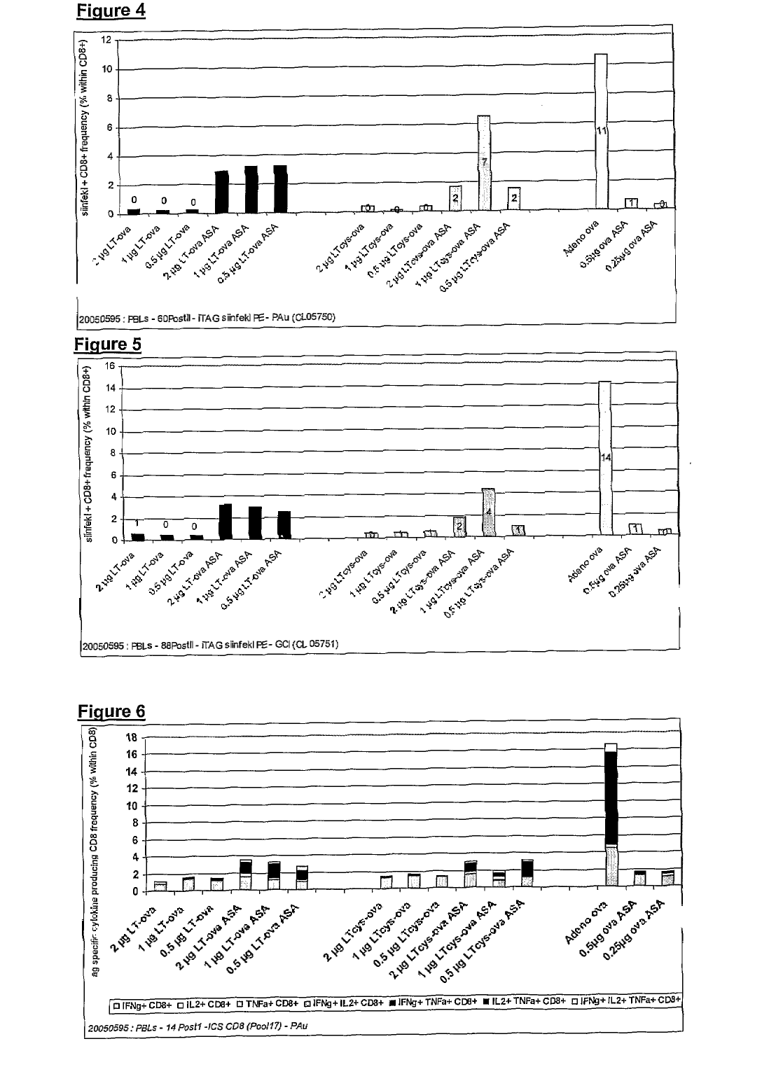

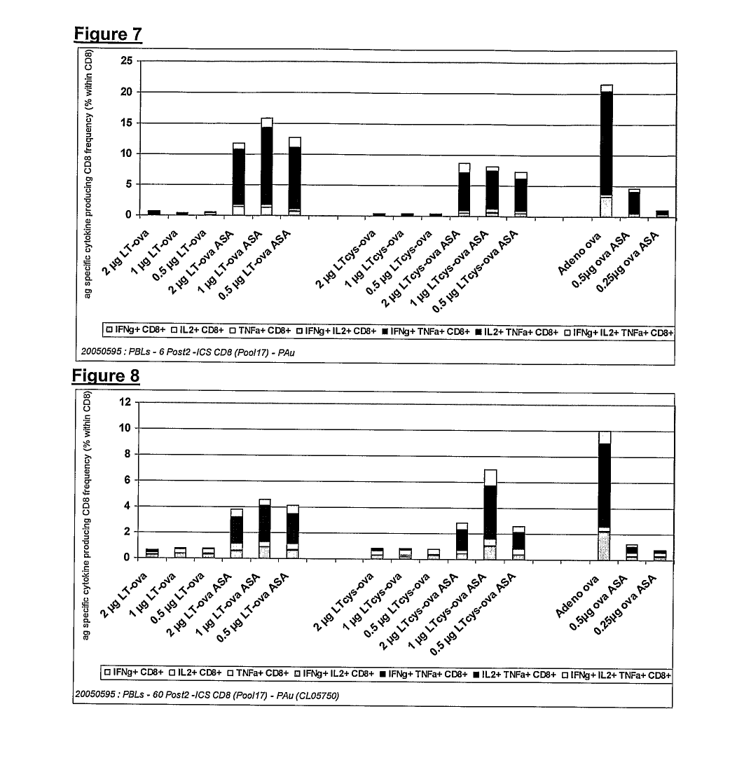

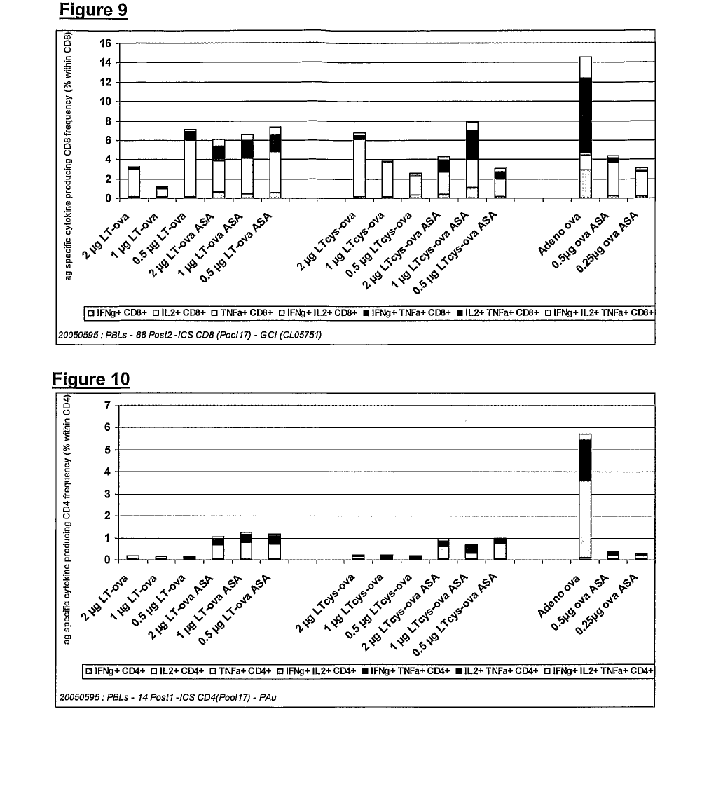

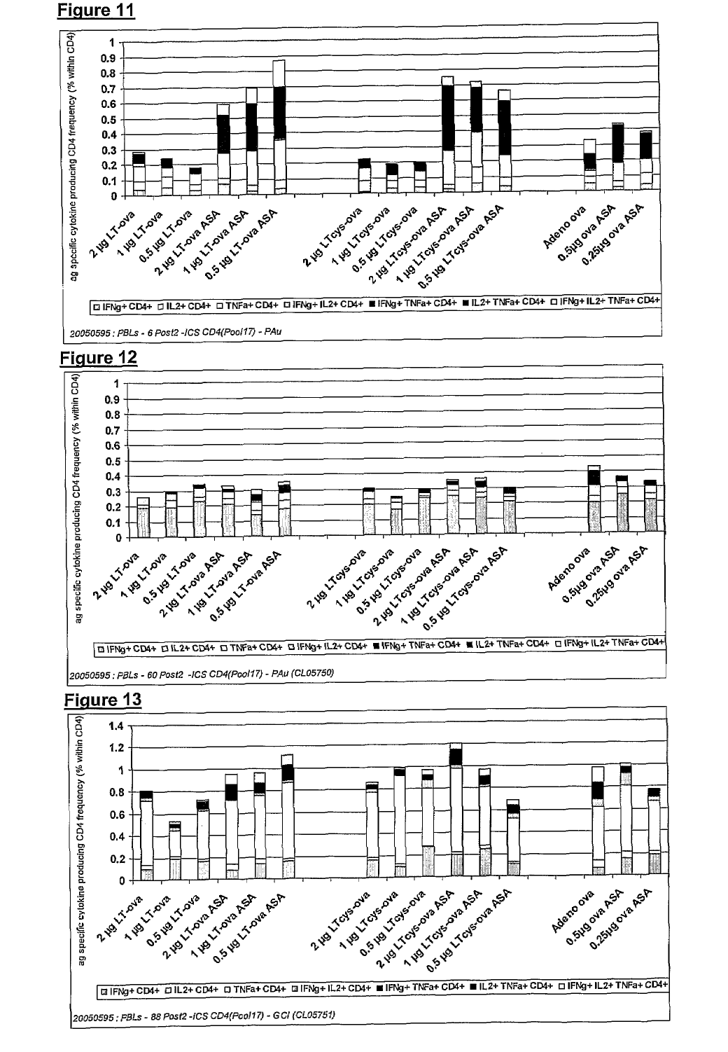

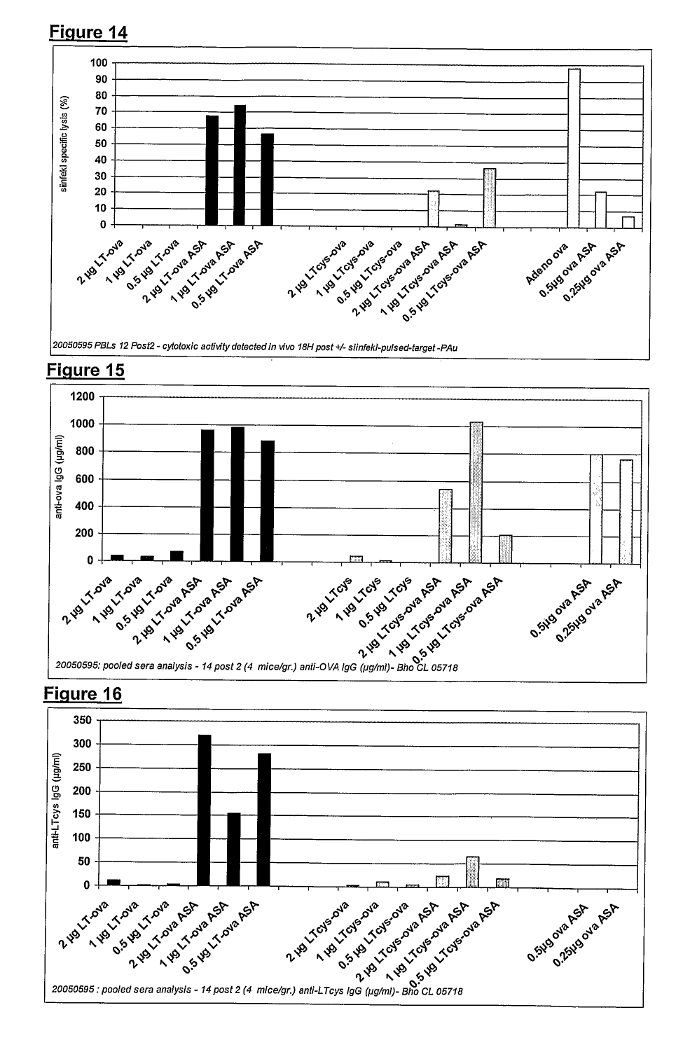

FIG. 1: CD8 response—tetramer 6 days post 1—siinfekl specific CD8 frequency (% within CD8+)

FIG. 2: CD8 response—tetramer 14 days post 1—siinfekl specific CD8 frequency (% within CD8+)

FIG. 3: CD8 response—tetramer 6 days post 2—siinfekl specific CD8 frequency (% within CD8+)

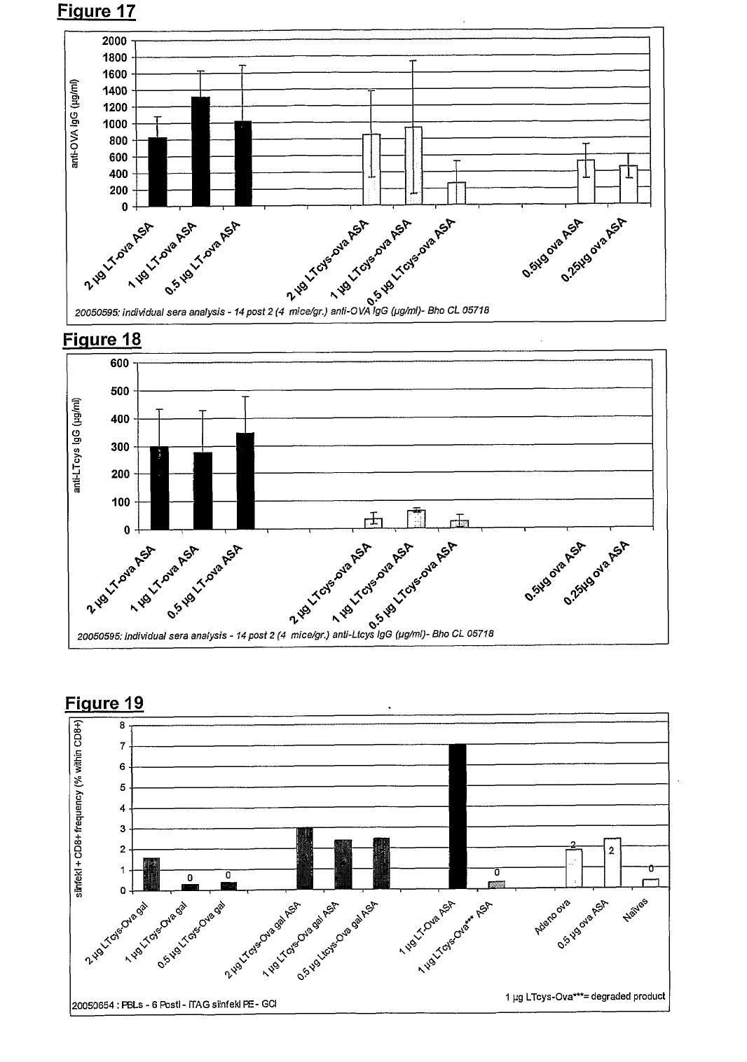

FIG. 4: CD8 response—tetramer 60 days post 2—siinfekl specific CD8 frequency (% within CD8+)

FIG. 5: CD8 response—tetramer 88 days post 2—siinfekl specific CD8 frequency (% within CD8+)

FIG. 6: CD8 response—ICS: 14 days post 1—ova specific cytokine producing CD8 frequency (% within CD8+)

FIG. 7: CD8 response—ICS: 6 days post 2—ova specific cytokine producing CD8 frequency (% within CD8+)

FIG. 8: CD8 response—ICS: 60 days post 2—ova specific cytokine producing CD8 frequency (% within CD8+)

FIG. 9: CD8 response—ICS: 88 days post 2—ova specific cytokine producing CD8 frequency (% within CD8+)

FIG. 10: CD4 response—ICS: 14 days post 1—ova specific cytokine producing CD4 frequency (% within CD4+)

FIG. 11: CD4 response—ICS: 6 days post 2—ova specific cytokine producing CD4 frequency (% within CD4+)

FIG. 12: CD4 response—ICS: 60 days post 2—ova specific cytokine producing CD4 frequency (% within CD4+)

FIG. 13: CD4 response—ICS: 88 days post 2—ova specific cytokine producing CD4 frequency (% within CD4+)

FIG. 14: CD8 response—cytotoxic activity detected in vivo 18H after target injection: 12 days post 2—siinfekl specific lysis (%)

FIG. 15: Humoral response—ELISA—pooled sera: anti-ova specific antibody titer (14 post2)

FIG. 16: Humoral response—ELISA —pooled sera: anti-LTcys specific antibody titer (14 post2)

FIG. 17: Humoral response—ELISA —individual sera: anti-ova specific antibody titer (14 post2)

FIG. 18: Humoral response—ELISA —individual sera: anti-LTcys specific antibody titer (14 post2)

The following Figures Show the Effect of Adjuvant System A on the Immune Response to Both LT-Ova and purified LTcys-Ova.

FIG. 19: CD8 response—tetramer 6 days post 1—siinfekl specific CD8 frequency (% within CD8+)

FIG. 20: CD8 response—tetramer 14 days post 1—siinfekl specific CD8 frequency (% within CD8+)

FIG. 21: CD8 response—tetramer 6 days post 2—siinfekl specific CD8 frequency (% within CD8+)

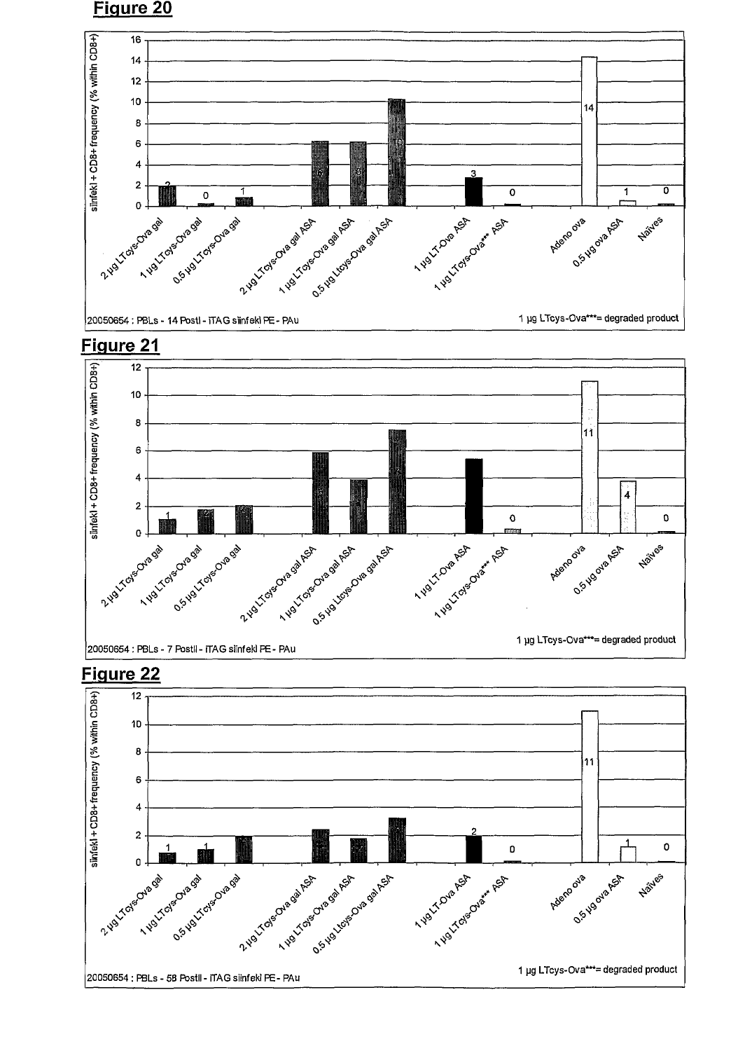

FIG. 22: CD8 response—tetramer 58 days post 2—siinfekl specific CD8 frequency (% within CD8+)

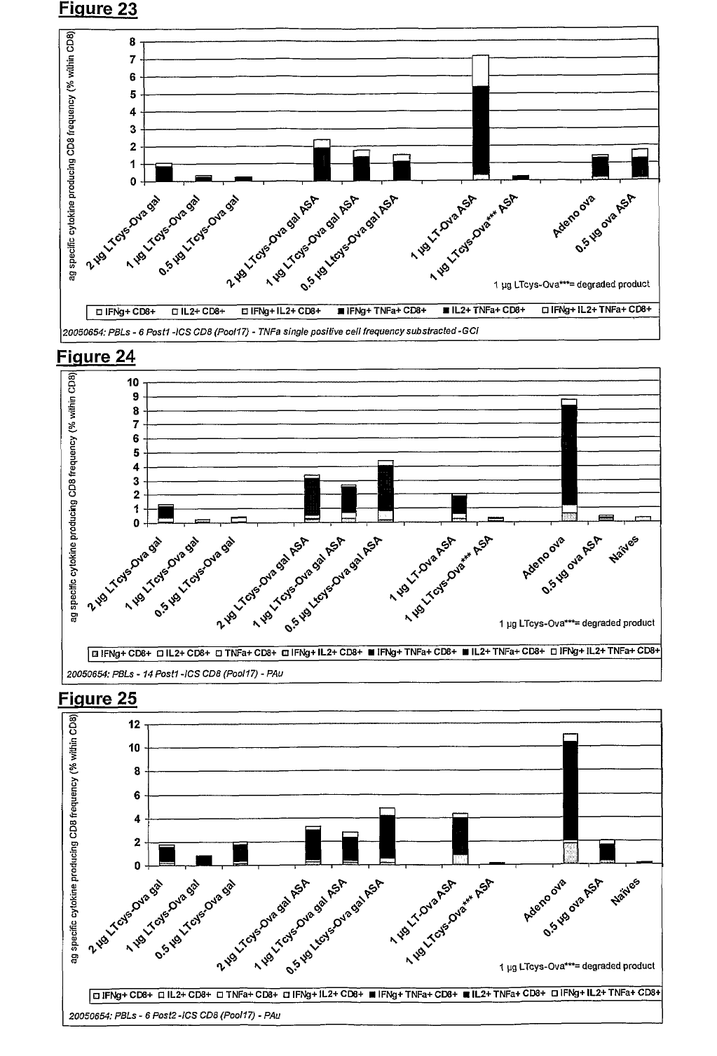

FIG. 23: CD8 response—ICS: 6 days post 1—ova specific cytokine producing CD8 frequency (% within CD8+)

FIG. 24: CD8 response—ICS: 14 days post 1—ova specific cytokine producing CD8 frequency (% within CD8+)

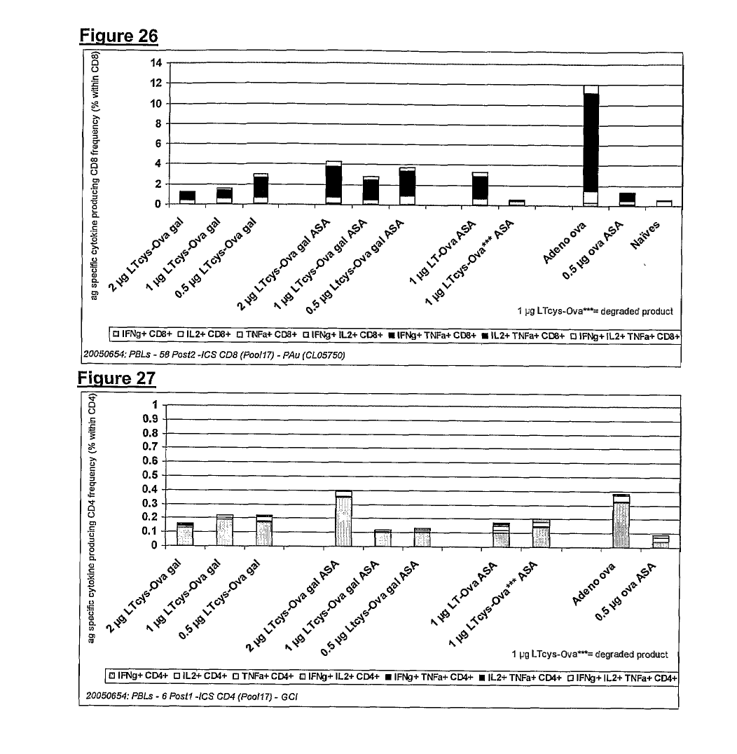

FIG. 25: CD8 response—ICS: 6 days post 2—ova specific cytokine producing CD8 frequency (% within CD8+)

FIG. 26: CD8 response—ICS: 58 days post 2—ova specific cytokine producing CD8 frequency (% within CD8+)

FIG. 27: CD4 response—ICS: 6 days post 1—ova specific cytokine producing CD4 frequency (% within CD4+)

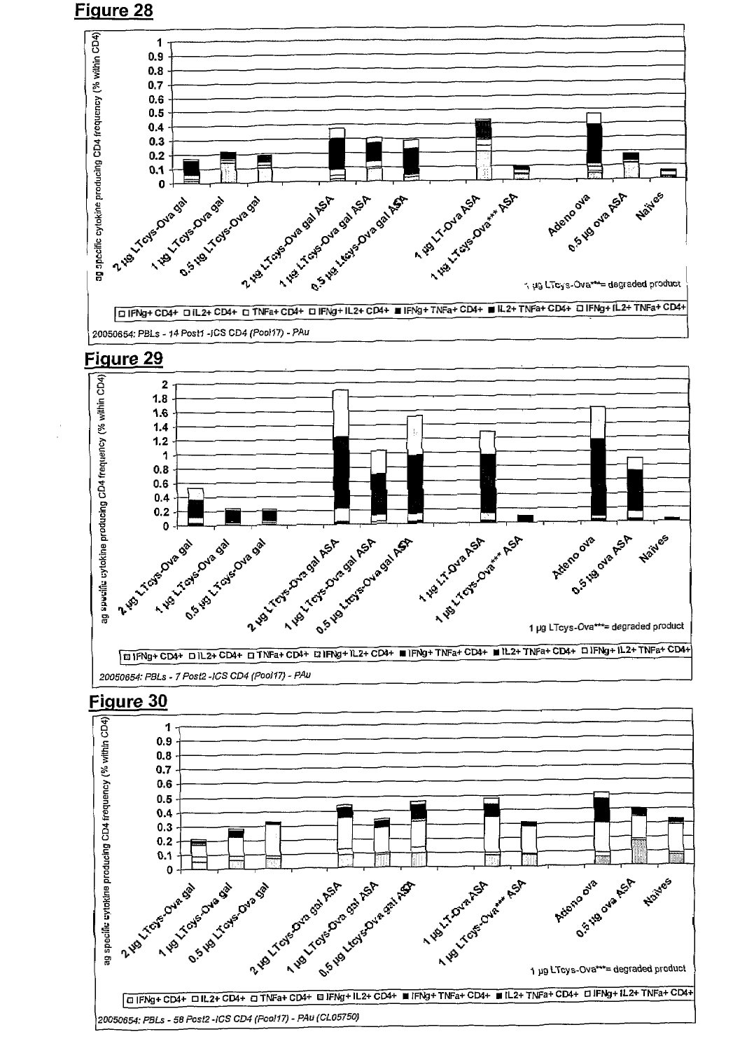

FIG. 28: CD4 response—ICS: 14 days post 1—ova specific cytokine producing CD4 frequency (% within CD4+)

FIG. 29: CD4 response—ICS: 6 days post 2—ova specific cytokine producing CD4 frequency (% within CD4+)

FIG. 30: CD4 response—ICS: 58 days post 2—ova specific cytokine producing CD4 frequency (% within CD4+)

FIG. 31: CD8 response—cytotoxic activity detected in vivo 18H after target injection: 21 days post 1—siinfekl specific lysis (%)

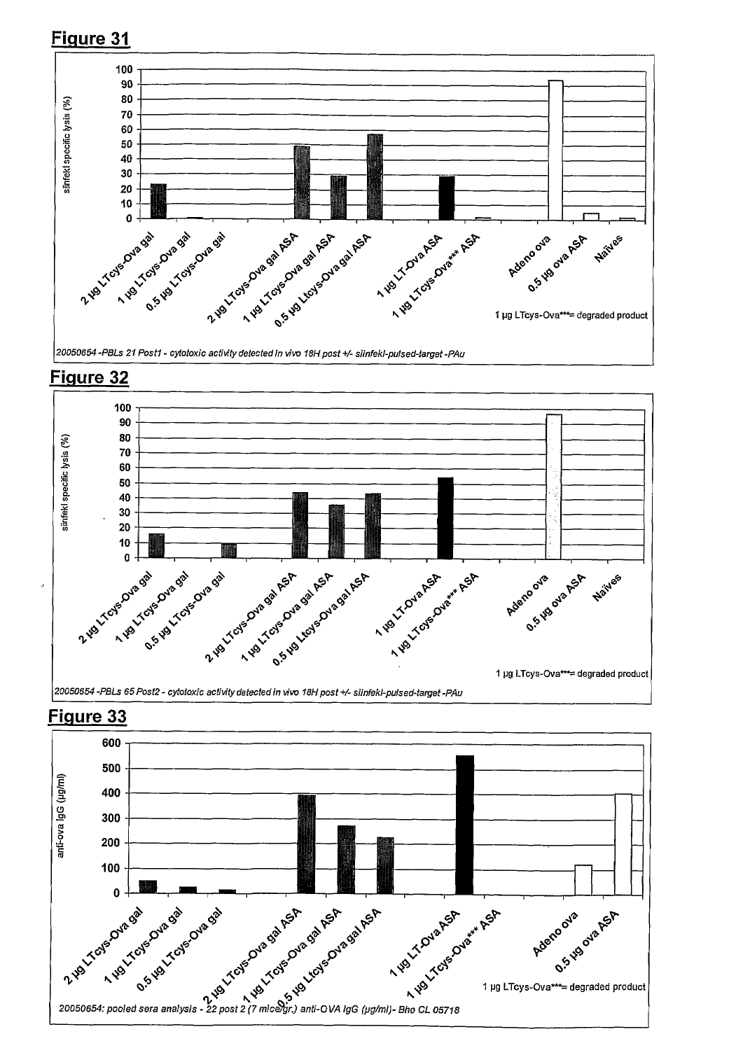

FIG. 32: CD8 response—cytotoxic activity detected in vivo 1 8H after target injection: 65 days post 2—siinfekl specific lysis (%)

FIG. 33: Humoral response—ELISA —pooled sera: anti-ova specific antibody titer (22 post2)

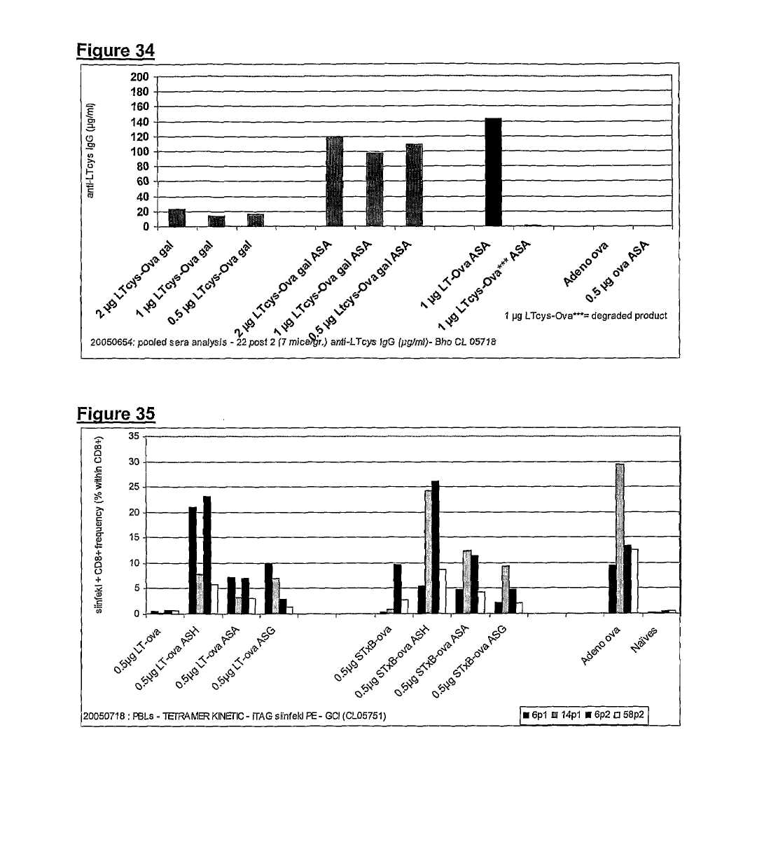

FIG. 34: Humoral response—ELISA —pooled sera: anti-LTcys specific antibody titer (22 post2)

The Following Figures Show the Effect of Adjuvants Systems A,H and G on the Immune Response to StxB-Ova and LT-Ova.

FIG. 35: CD8 response kinetic—tetramer analysis at different time points (7post1, 14post1, 6post2, 58post2)—siinfekl specific CD8 frequency (% within CD8+)

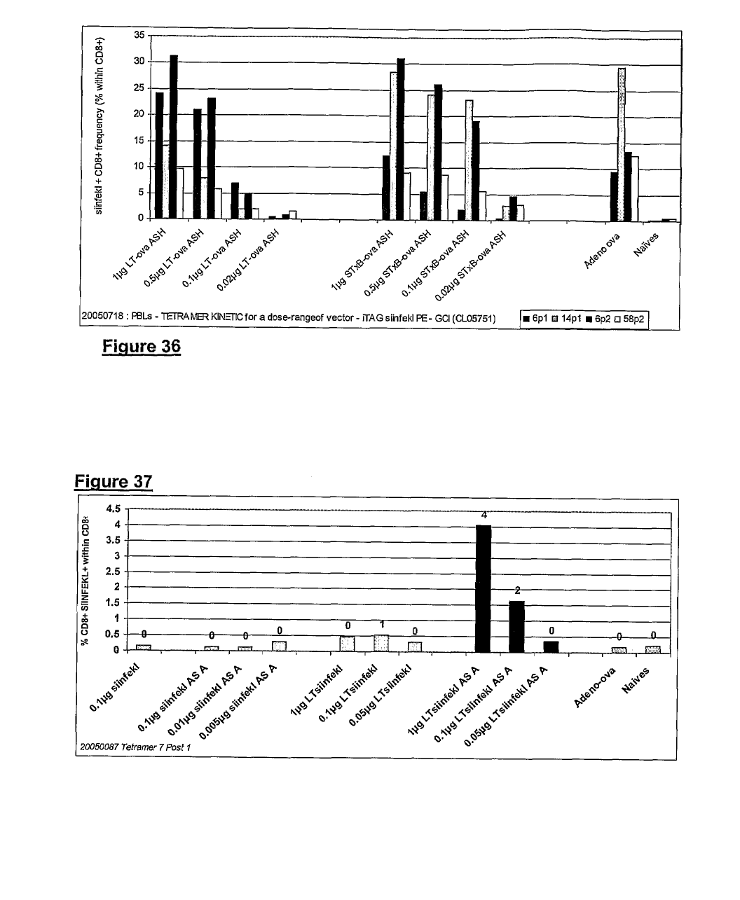

FIG. 36: CD8 response kinetic—tetramer analysis at different time points (7post1, 14post1, 6post2, 58post2) for a dose range of both vectors (LT vs STXB)—siinfekl specific CD8 frequency (% within CD8+)

The Following Figures Show the Effect of Adjuvant System A on the Immune Response Against Siinfekl Conjugated to Alternative Vectors.

FIG. 37: Siinfekl-specific CD 8 frequency in PBLs 7 days after primary injection with AS A LTSiinfekl vaccine

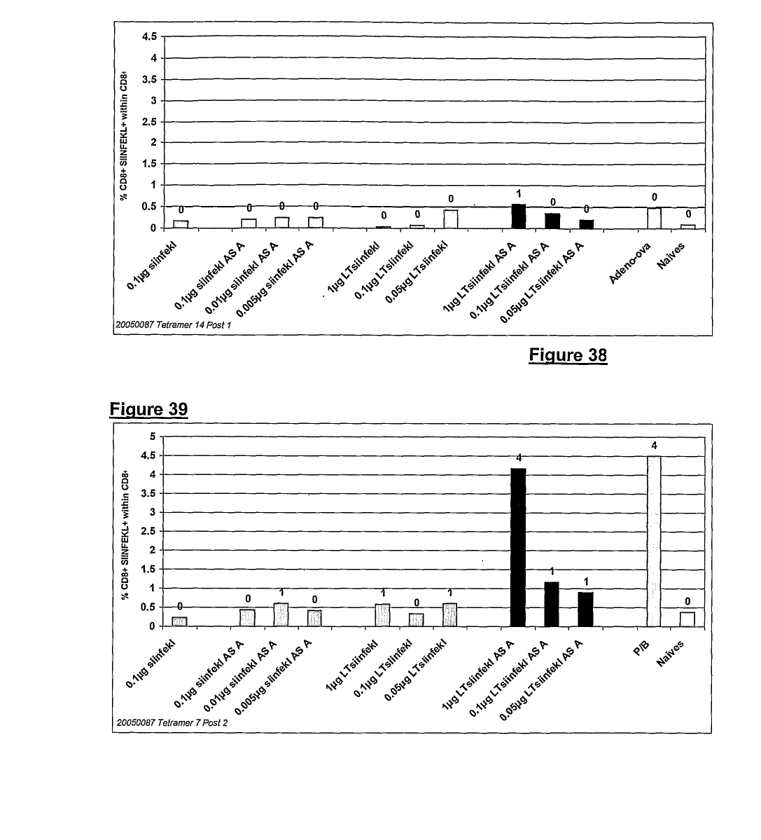

FIG. 38: Siinfekl-specific CD 8 frequency in PBLs 15 days after primary injection with AS A LTSiinfekl vaccine

FIG. 39: -Siinfekl-specific CD8 frequency in PBLs 7 days after second injection with AS A LTSiinfekl vaccine

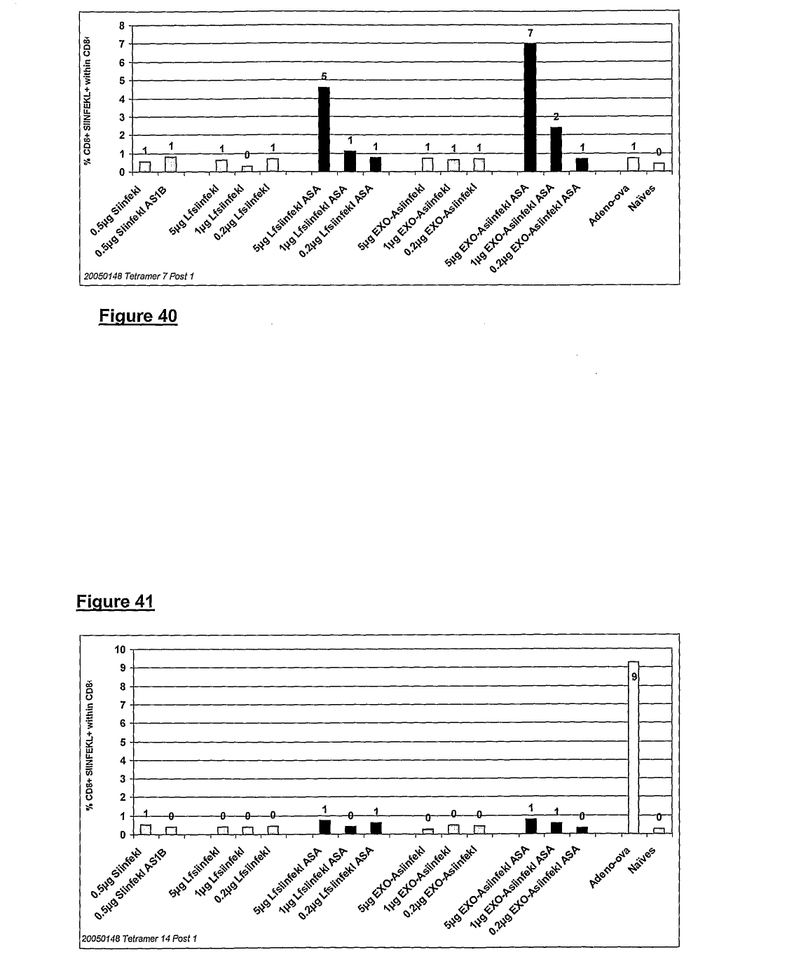

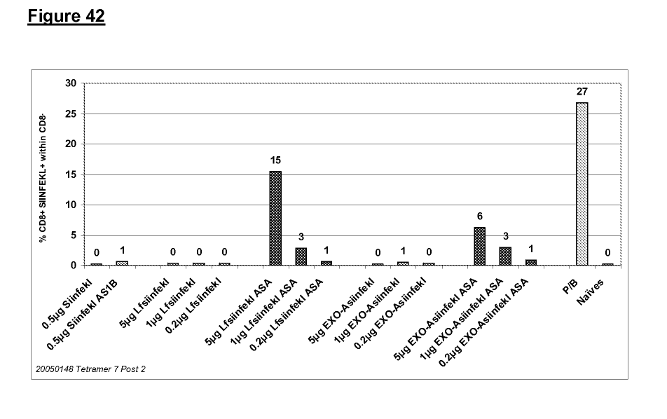

FIG. 40: Siinfekl-specific CD 8 frequency in PBLs 7 days after primary injection with Exo-A-Siinfekl AS A or LF-Siinfekl AS A vaccine

FIG. 41: Siinfekl-specific CD 8 frequency in PBLs 14 days after primary injection with Exo-A-Siinfekl AS A or LF-Siinfekl AS A vaccine

FIG. 42: -Siinfekl-specific CD8 frequency in PBLs 7 days after second injection with Exo-A-Siinfekl AS A or LF-Siinfekl AS A vaccine

### EXAMPLE 1

### Reagents and Media

### 1.1 Preparation of LTB, LTB-cys and LTB-siinfekl Recombinants

The LTB, LTB-cys (SEQ ID NO.7) and LTB-siinfekl (SEQ ID NO. 8) coding sequences were amplified by PCR and cloned into pET expression vectors for expression in E Coli. A total protein extract was obtained from a bacterial pellet at OD₍₆₂₀₎ 60 using the French press. After 30′ centrifugation at 15000 g, the supernatant was harvested and precipitated by adding (NH4)₂SO4 (4.95 g/10 ml) and incubating at least 4 hours at 4° C. The protein pellet was harvested after centrifugation, dissolved in PBS (4 times concentration), and dialyzed intensively against the same buffer. The insoluble fraction was eliminated by centrifugation and 0.22 μm filtration. The clarified supernatant was loaded on a XK1 6/15cm length column containing 15 ml PBS pre-equilibrated immobilized D-galactose resin (Calbiochem), and washed with PBS until the optical density dropped to basal level. The bound protein was eluted with 1 M galactose in PBS. After dialysis, the recovered protein is visualized by SDS Page, Coomassie staining and Western blotting. This method of purifying proteins using a D-galactose resin may also be used to determine whether a protein of interest binds the GM1 receptor.

SEQ ID NO.7

ATGAATAAAGTAAAATGTTATGTTTTATTTACGGCGTTACTATCCTCTCT
ATGTGCATACGGAGCTCCCCAGTCTATTACAGAACTATGTTCGGAATATC
GCAACACACAAATATATACGATAAATGACAAGATACTATCATATACGGAA
TCGATGGCAGGCAAAAGAGAAATGGTTATCATTACATTTAAGAGCGGCGC
AACATTTCAGGTCGAAGTCCCGGGCAGTCAACATATAGACTCCCAAAAAA
AAGCCATTGAAAGGATGAAGGACACATTAAGAATCACATATCTGACCGAG
ACCAAAATTGATAAATTATGTGTATGGAATAATAAAACCCCCAATTCAAT
TGCGGCAATCAGTATGGAAAACTGCTAA

SEQ ID NO.8

ATGAATAAAGTAAAATQTTATGTTTTATTTACGGCGTTACTATCCTCTCT
ATGTGCATACGGAGCTCCCCAGTCTATTACAGAACTATGTTCGGAATATC
GCAACACACAAATATATACGATAAATGACAAGATACTATCATATACGGAA
TCGATGGCAGGCAAAAGAGAAATGGTTATCATTACATTTAAGAGCGGCGC
AACATTTCAGGTCGAAGTCCCGGGCAGTCAACATATAGACTCCCAAAAAA
AGCCATTGAAAGGATGAAGGACACATTAAGAATCACATATCTGACGGAGA
CCAAAATTGATAAATTATGTGTATGGAATAATAAAACCCCCAATTCAATT
GCGGCAATCAGTATGGAAAACAGCCAGCTTGAGAGTATAATCAACTTTGA
AAAACTGACTGAATGGCGCGGCCGCTAG

The LTB and LTB-cys vector (SEQ ID NO. 7) were conjugated to the commercially available full-length chicken Ovalbumin antigen as described in the following sections and formulated in either ASA, ASH or ASG.

The LTB-siinfekl (SEQ ID NO. 8) recombinant was directly formulated in adjuvant system A noted below.

### Preparation of LTB/OVA Conjugate

The commercially available full-length chicken Ovalbumin antigen (5 mg) was reduced to expose SH groups by DTT treatment for 2 hours at room temperature. DTT was removed using a PD10 (Sephadex G-25, Amersham) column (elution with 2 mM phosphate buffer pH 6.8, fractions of 1 ml). The LTB vector described above (8 mg) was activated using a 10-fold molar excess of SGMBS for 1 hour at room temperature. The excess of SGMBS was removed using a PD10 column (elution with 100 mM phosphate buffer pH 7.2, fractions of 1 ml).

For conjugation, equimolar amounts of reduced Ovalbumin (OVA-SH) and activated LTB were reacted for 1 hour at room temperature. The resulting conjugate was purified by molecular filtration on a S-300 HR Sephacryl column (elution with 100 mM Phosphate buffer pH 6.8, fractions of 1 ml).

The LTB/OVA conjugate was then formulated in adjuvant system A noted below. This product is indicated as LT-ova on graphs.

### Preparation of LTB-cys/OVA Conjugate

The commercially available full-length chicken Ovalbumin antigen (10 mg) was activated using a 80-fold molar excess of SGMBS for 1 hour at room temperature.

The excess of SGMBS was removed using a PD10 column (elution with 1 ml fractions of DPBS buffer (NaCl 136.87 mM, KCl 2.68 mM, Na2HPO4 8.03 mM, KH2PO4 1.47 mM pH 7.5), fractions of 1 ml).

For conjugation, equimolar amounts of LTB-cys and activated Ovalbumin were reacted for 1 hour at room temperature. The resulting conjugate was purified by molecular filtration on a S-300 HR Sephacryl column (elution with DPBS buffer, fractions of 4 ml).

The LTB-cys/OVA conjugate was then formulated in adjuvant system A noted below. This product is indicated as LTcys-ova on graphs.

Method to Purify the LTB Subunit from E. coli Lysate:

1 I bacterial pellet OD ₍₆₂₀₎ 50 in DPBS w/o CaMg buffer is extract by French press; After 30′ centrifugation 5000 g, supernatant is harvested and treated with 50000 u benzonase 1 h RT. Insoluble fraction is eliminated by centrifugation 30′ 15000g and 0.22 μm filtration. Clarified supernatant is loaded on XK16/20 column containing 20 ml DPBS w/o CaMg buffer pre-equilibrated immobilized galactose resin, and washed with same buffer till OD drops to basal level. LTB is eluated by 1 M galactose in DPBS w/o CaMg buffer. Finely, LTB is dialysed intensively against DPBS w/o CaMg buffer.

Endotoxins are removed by Acticlean resin incubation.

### Preparation of Adjuvanted STxB-Ova

STxB coupled to full length Chicken ovalbumin: to allow the chemical coupling of proteins to a defined acceptor site in STxB, a cysteine was added to the C-terminus of the wild-type protein, yielding STxB-Cys. The recombinant mutant STxB-Cys protein was produced as previously described (Haicheur et al.; 2000, J. Immunol. 165, 3301). Endotoxin concentration determined by the Limulus assay test was below 0.5 EU/ml. STxB-ova has been previously described (HAICHEUR et al., 2003, Int. Immunol., 15, 1161-1171)and was kindly provided by Ludger Johannes and Eric Tartour (Curie Institute).

### 1.2 Preparation of LFn-siinfekl and LFn-OVA¹⁶¹⁻²⁹¹ Recombinants

Two synthetic genes were prepared that contained the amino terminal 255 amino acids from Anthrax LF toxin flanked by a 6×His tail and either the Siinfekl coding sequence or a larger Ovalbumin fragment containing this epitope (fragment 161-291) (SEQ ID No. 9 and 10, respectively). The resulting products were cloned into a pET expression vector for expression in E Coli. Cells were recovered by centrifugation, concentrated (25 to 40×) and lysed using a French press. Aggregates were dissociated in 6 M urea overnight at 4° C. To purify the recombinant proteins, 5 ml of previously equilibrated Ni-NTA resin (Qiagen) were added to the lysate, incubated 2 hours at 4° C. on a rotating wheel and loaded onto a polyprep disposable column (BioRad). The column was washed three times with 15 ml of a 300 mM NaCl, 6 M urea, 5 mM imidazole, 50 mM Phosphate buffer pH8 before elution with 4×2 ml of the same buffer containing 500 mM imidazole The recovered proteins were visualized by SDS Page, Coomassie staining and Western blotting, and urea was removed by dialysis.

SEQ ID No.9

| | |
|---|---|
| ATGGGCCACCATCACCATCACCATTCTTCTGGTGCGGGCG | 40 |
| GTCATGGTGATGTAGGTATGCACGTAAAAGAGAAAGAGAA | 80 |
| AAATAAAGATGAGAATAAGAGAAAAGATGAAGAACGAAAT | 120 |
| AAAACACAGGAAGAGCATTTAAAGGAAATCATGAAACACA | 160 |
| TTGTAAAAATAGAAGTAAAAGGGGAGGAAGCTGTTAAAAA | 200 |
| AGAGGCAGCAGAAAAGCTACTTGAGAAAGTACCATCTGAT | 240 |
| GTTTTAGAGATGTATAAAGCAATTGGAGGAAAGATATATA | 280 |
| TTGTGGATGGTGATATTACAAAACATATATCTTTAGAAGC | 320 |
| ATTATCTGAAGATAAGAAAAAAATAAAAGACATTTATGGG | 360 |
| AAAGATGCTTTATTACATGAACATTATGTATATGCAAAAG | 400 |
| AAGGATATGAACCCGTACTTGTAATCCAATCTTCGGAAGA | 440 |
| TTATGTAGAAAATACTGAAAAGGCACTGAACGTTTATTAT | 480 |
| GAAATAGGTAAGATATTATCAAGGGATATTTTAAGTAAAA | 520 |
| TTAATCAACCATATCAGAAATTTTTAGATGTATTAAATAC | 560 |
| CATTAAAAATGCATCTGATTCAGATGGACAAGATCTTTTA | 600 |
| TTTACTAATCAGCTTAAGGAACATCCCACAGACTTTTCTG | 640 |
| TAGAGTTCTTGGAACAAAATAGCAATGAGGTACAAGAAGT | 680 |
| ATTTGCGAAAGCTTTTGCATATTATATCGAGCCACAGCAT | 720 |
| CGTGATGTTTTACAGCTTTATGCACCGGAAGCTTTTAATT | 760 |
| ACATGGATAAATTTAACGAACAAGAAATAAATCTATCCGG | 800 |
| ATCCCAGCTTGAGAGTATAATCAACTTTGAAAAACTGACT | 840 |
| GAATGGTGA | 849 |

SEQ ID No.10

| | |
|---|---|
| ATGGGCCACCATGACCATCACCATTCTTCTGGTGCGGGCG | 40 |
| GTCATGGTGATGTAGGTATGCACGTAAAAGAGAAAGAGAA | 80 |
| AAATAAAGATGAGAATAAGAGAAAAGATGAAGAACGAAAT | 120 |
| AAAACACAGGAAGAGCATTTAAAGGAAATCATGAAACACA | 160 |
| TTGTAAAAATAGAAGTAAAAGGGGAGGAAGCTGTTAAAAA | 200 |
| AGAGGCAGCAGAAAAGCTACTTGAGAAAGTACCATCTGAT | 240 |
| GTTTTAGAGATGTATAAAGCAATTGGAGGAAAGATATATA | 280 |
| TTGTGGATGGTGATATTACAAAACATATATCTTTAGAAGC | 320 |
| ATTATCTGAAGATAAGAAAAAAATAAAAGACATTTATGGG | 360 |
| AAAGATGCTTTATTACATGAACATTATGTATATGCAAAAG | 400 |
| AAGGATATGAACCCGTACTTGTAATCCAATCTTCGGAAGA | 440 |
| TTATGTAGAAAATACTGAAAAGGCACTGAACGTTTATTAT | 480 |
| GAAATAGGTAAGATATTATCAAGGGATATTTTAAGTAAAA | 520 |
| TTAATCAACCATATCAGAAATTTTTAGATGTATTAAATAC | 560 |
| CATTAAAAATGCATCTGATTCAGATGGACAAGATCTTTTA | 600 |
| TTTACTAATCAGCTTAAGGAACATCCCACAGACTTTTCTG | 640 |
| TAGAGTTCTTGGAACAAAATAGCAATGAGGTACAAGAAGT | 680 |
| ATTTGCGAAAGCTTTTGCATATTATATCGAGCCACAGCAT | 720 |
| CGTGATGTTTTACAGCTTTATGCACCGGAAGCTTTTAATT | 760 |
| ACATGGATAAATTTAACGAACAAGAAATAAATCTATCCGG | 800 |
| ATCCGTCCTTCAGCCAAGCTCCGTGGATTCTCAAACTGCA | 840 |
| ATGGTTCTGGTTAATGCCATTGTCTTCAAAGGACTGTGGG | 880 |
| AGAAAACATTTAAGGATGAAGACACAGAAGCAATGCCTTT | 920 |
| CAGAGTGACTGAGCAAGAAAGCAAACCTGTGCAGATGATG | 960 |
| TACCAGATTGGTTTATTTAGAGTGGCATCAATGGCTTCTG | 1000 |
| AGAAAATGAAGATCCTGGAGCTTCCATTTGCCAGTGGGAC | 1040 |
| AATGAGCATGTTGGTGCTGTTGCCTGATGAAGTCTCAGGC | 1080 |
| CTTGAGCAGCTTGAGAGTATAATCAACTTTGAAAAACTGA | 1120 |
| CTGAATGGACCAGTTCTAATGTTATGGAAGAGAGGAAGAT | 1160 |
| CAAAGTGTACTTACCTCGCATGAAGATGGAGGAAAAATGA | 120 |

The LFn-siinfekl (SEQ ID No. 9) and LFn-OVA¹⁶¹⁻²⁹¹ (SEQ ID No. 10) recombinants were then formulated in adjuvant system A noted below.

LFn-siinfekl is indicated as LFsiinfekl is the graphs.

### 1.3 Preparation of ExoA-siinfekl Recombinant

A synthetic gene was prepared (Seq ID No. 11—see below) that corresponds to a non toxic form of the Exotoxin A (deletion of E553), in which the Siinfekl epitope is introduced so that it replaces most of the lb domain the toxin. The resulting product was cloned into a pET expression vector and expressed in E. coli. The recombinant protein was then extracted from inclusion bodies and purified essentially as described in FitzGerald et al., J Biol Chem 273, 9951, 1998.

SEQ ID NO.11

ATGGCCGAGGAAGCCTTCGACCTCTGGAACGAATGCGCCAAAGCCTGCGT
GCTCGACCTCAAGGACGGCGTGCGTTCCAGCCGCATGAGCGTCGACCCGG
CCATCGCCGACACCAACGGCCAGGGCGTGCTGCACTACTCCATGGTCCTG
GAGGGCGGCAACGACGCGCTCAAGCTGGCCATCGACAACGCCCTCAGCAT
CACCAGCGACGGCCTGACCATCCGCCTCGAAGGCGGCGTCGAGCCGAACA
AGCCGGTGCGCTACAGCTACACGCGCCAGGCGCGCGGCAGTTGGTCGCTG
AACTGGCTGGTACCGATCGGCCACGAGAAGCCCTCGAACATCAAGGTGTT
CATCCACGAACTGAACGCCGGCAACCAGCTCAGCCACATGTCGCCGATCT
ACACCATCGAGATGGGCGACGAGTTGCTGGCGAAGCTGGCGCGCGATGCC
ACCTTCTTCGTCAGGGCGCACGAGAGCAACGAGATGCAGCCGACGCTCGC
CATCAGCCATGCCGGGGTCAGCGTGGTCATGGCCCAGACCCAGCCGCGCC
GGGAAAAGCGCTGGAGCGAATGGGCCAGCGGCAAGGTGTTGTGCCTGCTC
GACCCGCTGGACGGGGTCTACAACTACCTCGCCCAGCAACGCTGCAACCT
CGACGATACCTGGGAAGGCAAGATCTACCGGGTGCTCGCCGGCAACCCGG
CGAAGCATGACCTGGACATCAAACCCACGGTCATCAGTCATCGCCTGCAC
TTTCCCGAGGGCGGCAGCCTGGCCGCGCTGACCGCGCACCAGGCTTGCCA
CCTGCCGCTGGAGACTTTCACCCGTCATCGCCAGCCGCGCGGCTGGGAAC
AACTGGAGCAGTGCGGCTATCCGGTGCAGCGGCTGGTCGCCCTCTACCTG
GCGGCGCGGCTGTCGTGGAACCAGGTCGACCAGGTGATCCGCAACGCCCT
GGCCAGCCCCGGCAGCGGCGGCGACCTGGGCGAAGCGATCCGCGAGCAGC
CGGAGCAGGCCCGTCTGGCCCTGACCCTGGCCGCCGCCGAGAGCGAGCGC
TTCGTCCGGCAGGGCACCGGCAACGACGAGGCCGGCGCGGCCAACCTGCA
CTGCCAGCTTGAGAGTATAATCAACTTTGAAAAACTGACTGAATGGTGCA
TGCAGGGCCCGGCGGACAGCGGCGACGCCCTGCTGGAGCGCAACTATCCC
ACTGGCGCGGAGTTCCTCGGCGACGGCGGCGACGTCAGCTTCAGCACCCG
CGGCACGCAGAACTGGACGGTGGAGCGGCTGCTCCAGGCGCACCGCCAAC
TGGAGGAGCGCGGCTATGTGTTCGTCGGCTACCACGGCACCTTCCTCGAA
GCGGCGCAAAGCATCGTCTTCGGCGGGGTGCGCGCGCGCAGCCAGGACCT
CGACGCGATCTGGCGCGGTTTCTATATCGCCGGCGATCCGGCGCTGGCCT
ACGGCTACGCCCAGGACCAGGACCCGACGCACGCGGCCGGATCCGCAACG
GTGCCCTGCTGCGGGTCTATGTGCCGCGCTCGAGCCTGCCGGGCTTCTAC
CGCACCAGCCTGACCCTGGCCGCGCCGGAGGCGGCGGGCGAGGTCGAACG
GCTGATCGGCCATCCGCTGCCGCTGCGCCTGGACGCCATCACCGGCCCCG
AGGAGGAAGGCGGGCGCCTGACCATTCTCGGCTGGCCGCTGGCCGAGCGC
ACCGTGGTGATTCCCTCGGCGATCCCCACCGACCCGCGCAACGTCGGCGG
CGACCTCGACCCGTCCAGCATCCCCGACAAGGAACAGGCGATCAGCGCCC
TGCCGGACTACGCCAGCCAGCCCGGCAAACCGCCGCGCGAGGACCTGAAG
TGA

The ExoA-siinfekl recombinant (SEQ ID No. 11) was then formulated in adjuvant system A noted below.

### 1.4 Galactose Binding Assay

The GM1 receptor preferentially recognized by the B subunit of the LT toxins is a cell surface monosialoganglioside (Gal(β1-3)GalNAc(β1-4)(NeuAc(α2-3))Gal(β1-4)Glc(β1-1)ceramide), where Gal is Galactose, GalNAc is N-acetylgalactosamine, NeuAc is acetylneuraminic acid and Glc is Glucose. The method described below involves an affinity chromatography on a commercially available galactose-linked agarose gel (Pierce). Galactose is the terminal carbohydrate portion of the oligosacharide moiety of G_{M1} and is thought to represent the minimal structure recognized by the B subunit of the LT toxin (Sixma et al. Nature 355 (1992), p. 561). This method is used to purify the B subunit of the LT toxin directly from E. coli lysate (see below): it can therefore be assumed that the galactose binding assay can be used to identify proteins that bind the GM1 receptor.

The protein of interest (in DPBS w/o CaMg buffer) is loaded by pumping on a XK16/20 column (Amersham Biosciences) packed with 12 ml of immobilized D-Galactose resin (Pierce) previously equilibrated in the same buffer. At least 3 bed volumes of DPBS w/o CaMg buffer are then passed through the column at the operating flow rate of 0.5 ml/min. After washing, the bound protein is eluated from the resin with a flow of D-galactose 1 M (in DPBS w/o CaMg buffer). The 1-ml fractions collected during washing and elution are analyzed by SDS page, Coomassie staining and Western blotting. These analytical techniques allow identification of whether the protein is bound to the galactose, and hence will bind the GM1 receptor. The fractions containing the protein of interest can be pooled and dialyzed against DPBS w/o CaMg buffer to remove the D-galactose.

### 1.5 Galabiose Binding Assay

The Gb3 receptor preferentially recognized by the B subunit of Shiga toxin is a cell surface glycosphingolipid, globotriaosylceramide (Galα1-4Galβ1-4 glucosylceramide), where Gal is Galactose. The method described below is based on that described by Tarrago-Trani (Protein Extraction and Purification 38, pp 170-176, 2004), and involves an affinity chromatography on a commercially available galabiose-linked agarose gel (calbiochem). Galabiose (Galα1->4Gal) is the terminal carbohydrate portion of the oligosacharide moiety of Gb3 and is thought to represent the minimal structure recognized by the B subunit of Shiga toxin. This method has been successfully used to purify Shiga toxin directly from E. coli lysate. Therefore it can be assumed that proteins that bind this moiety will bind the Gb3 receptor.

The protein of interest in PBS buffer (500 μl) is mixed with 100 μl of immobilised galabiose resin (Calbiochem) previously equilibrated in the same buffer, and incubated for 30 min to 1 hour at 4° C. on a rotating wheel. After a first centrifugation at 5000 rpm for 1 min, the pellet is washed twice with PBS. The bound material is then eluated twice by re-suspending the final pellet in 2×500 μl of 100 mM glycine pH 2.5. Samples corresponding to the flow-through, the pooled washes and the pooled eluates are then analyzed by SDS Page, Coomassie staining and Western blotting. These analytical techniques allow identification of whether the protein is bound to the galabiose, and hence will bind the Gb3 receptor.

### 1.6 Preparation of Adjuvant Systems

### 1.6.1 Preparation of Adjuvant System A: QS21 and 3D-MPL.

A mixture of lipid (such as phosphatidylcholine either from egg-yolk or synthetic) and cholesterol and 3 D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 5 to 50 mg/ml.

The liposomes have a size around 100 nm. The liposomes by themselves are stable over time and have no fusogenic capacity. Sterile bulk of liposomes was mixed with QS21 in aqueous solution with a chol/QS21 ratio equal to 5/1 (w/w). This mixture is referred as DQMPLin. DQMPLin is then diluted in PBS to reach a final concentration of 10 μg/ml of 3D-MPL. PBS composition was PO4: 50 mM; NaCl: 100 mM pH 6.1 . . . Non-live vector was then added. Between each addition of component, the intermediate product was stirred for 5 minutes. The pH was checked and adjusted if necessary to 6.1+/−0.1 with NaOH or HCl.

Injection volume of 50 μl corresponded to a determined antigen dose (high dose described in the table below), 0.5 μg of 3 D-MPL and QS21 and 5 μg of CpG. These formulations were then diluted in a solution of 3D-MPL and QS21 (at a concentration of 10 and 10 μg/ml respectively) to obtain antigen dose-ranges as described in the table.

| | antigen Dose-range | MPL | QS21 | Shown |
|---|---|---|---|---|
| Antigen | (μg) | (μg) | (μg) | in figure |
| LF-OVA(161-291) | 5, 1 and 0.2 | 0.5 | 0.5 | |
| LF-Siinfekl | 5, 1 and 0.2 | 0.5 | 0.5 | 40 to 42 |
| LT_OVA | 2, 1 and 0.5 | 0.5 | 0.5 | 1 to 35 |
| LT-Cys-OVA | 2, 1 and 0.5 | 0.5 | 0.5 | 1 to 18 |
| LT-Cys-OVA-Gal | 2, 1 and 0.5 | 0.5 | 0.5 | 19 to 34 |
| ExoA-Siinfekl | 5, 1 and 0.2 | 0.5 | 0.5 | 40 to 42 |
| LT-Siinfekl | 1, 0.1 and 0.05 | 0.5 | 0.5 | 39 |

1.6.2 Adjuvant system G: CpG2006

Sterile bulk CpG was added to PBS or NaCl 150 mM solution to reach a final concentration of 100 μg/ml.

Antigen was then added to reach a final concentration of 10 μg/ml.

The CpG used was a 24-mers with the following sequence 5′-TCG TCG TTT TGT CGT TTT GTC GTT-3′ (Seq ID No.4). Between each addition of component, the intermediate product was stirred for 5 minutes. The pH was checked and adjusted if necessary to 6.1+/−0.1 with NaOH or HCl.

Injection volume of 50 μl corresponded to 0.5 μg of each conjugated vectors (LT-OVA and StX-OVA) and 5 μg of CpG ( ).Data are shown in FIGS. 35 and 36

1.5.3 Adjuvant system H: QS21, 3D-MPL and CpG2006

Sterile bulk CpG was added to PBS solution to reach a final concentration of 100 μg/ml. PBS composition was PO₄: 50 mM; NaCl: 100 mM pH 6.1. Antigens were then added to reach a final concentration of 20 μg/ml. Finally, QS21 and 3 D-MPL were added as a premix of sterile bulk liposomes containing 3 D-MPL and QS21 referred as DQMPLin to reach final 3D-MPL and QS21 concentrations of 10 μg/ml. The CpG used was a 24-mers with the following sequence 5′-TCG TCG TTT TGT CGT TTT GTC GTT-3′ (Seq ID No.4). Between each addition of component, the intermediate product was stirred for 5 minutes. The pH was checked and adjusted if necessary to 6.1+/−0.1 with NaOH or HCl.

Injection volume of 50 μl corresponded to 1 μg of conjugated vectors (LT-OVA and STX-OVA), 0.5 μg of 3 D-MPL and QS21 and 5μg of CpG. These formulations were then diluted in a solution of 3D-MPL/QS21 and CpG (at a concentration of 10, 10 and 100 μg/ml respectively) to obtain doses of 0.5, 0.1 and 0.02 μg of antigen. (these formulations used for experiments shown in FIGS. 35 and 36)

### EXAMPLE 2

### Vaccination of C57/B6 Mice with Vaccines of the Invention

Formulations described above were used to vaccinate 6-8 week old C57BL/B6 (H2Kb), female mice (10/group). The mice received two injections spaced 14 days apart and were bled at specific time points between week 1 and week 12 as indicated on the graphs. The mice were vaccinated intramuscularly (injection into the left gastrocnemien muscle of a final volume of 50 μl) with ex-tempo formulation. The antigenic recombinant adenovirus was injected at a dose of 10⁸ to 5.10⁸ VP. At several time-points after immunization, various immunological read-outs were performed as described below.

### 2.1 Immunological Assays:

### Technology Principle

### Tetramer Principle:

Tetramer assay is the measure of the frequency of epitope-specific TCD8 by flow cytometry. This procedure has the advantage of analyzing lymphoid cells without any in vitro cultivation step. Lymphoid cells are incubated with an anti-CD8 antibody as well as with an peptide/MHC tetramer (consisting of immunodominant SIINFEKL peptides bound to H-2Kb tetramer, a complex capable of specific TCR binding), both fluor-labeled. Results are expressed as a frequency of tetramer+CD8+ T lymphocyte within the TCD8+ cell population.

### ICS Principle

ICS (Intracellular Cytokine Staining) is the technology which allow the quantification of antigen specific T lymphocytes on the basis of cytokine production.

Lymphoid cells are re-stimulated 18H in vitro with peptide(s) in the presence of a secretion inhibitor (brefeldin).

These cells are then processed by conventional immunofluorescent procedure using fluorescent antibodies (CD4, CD8, IFNg, IL2 and TNFa).

Results are expressed as a frequency of cytokine positive cell within CD4 and CD8 T-cells.

### CMC Principle:

CMC in vivo (Cell Mediated Cytotoxicity detected in vivo) is an assay that monitors the antigen specific cytotoxic activity without any manipulation of the effector cell. It relies on the IV injection of two CFSE labeled cell populations—control target cells and target cells pulsed with the MHC class I binding peptide derived from the antigen—in the blood stream of vaccinated animals (Aichele et al. 1997).

Targets are lymphoïd cells from naive mice that are labeled with 2 different concentrations of CFSE. 1 8H after injection of target cells, mice are sacrificed, blood sample from vaccinated mice are collected. PBLs are then analyzed using flow cytometry.

The percentage of cytotoxic activity is calculated by looking at the number of surviving antigen specific targets compared to the non specific control target. More precisely, FACS analysis on histogram give parameters such as M1 and M2 which are respectively the number of non specific targets (−) and specific targets (+).

Percentage of peptide specific lysis is calculated as follow:

$$lysis \% = 100 - ( \frac{corrected specific target ( + )}{non specific control target ( - )} \times 100 )$$

$$Corrected target += \frac{target +}{( preinj . + )} \times ( preinj . - )$$

Corrected target (+)=number of peptide pulsed target FACS acquired after injection in vivo, “normalised” as compared to preinjected target cells. preinj.=mix of peptide pulsed target (+) and non pulsed (−) FACS acquired before injection in vivo

### Detailed Protocols (Ova Model)

### Organ Collection

PBLs Isolation

Blood was taken from retro orbital vein (50 μl per mouse, 10 mice per group) and directly diluted in RPMI+heparin 1/10 (LEO) medium. PBLs were isolated through a lymphoprep gradient (CEDERLANE). Cells were then washed, counted and finally were re-suspended at ad hoc dilution in a ad hoc buffer (see below)

Spleen Cell Isolation

Briefly, total cells were extracted by disruption of spleen, cells are then resuspended within a large volume of RPMI (5 spleen within 35 ml). Spleen cells are isolated through a lymphoprep gradient (CEDERLANE). Lymphocytes are then washed, counted and finally re-suspended at ad hoc dilution in a ad hoc buffer (see below) washed.

Lymph Node Cell Isolation

Briefly, total cells are extracted by disruption of the draining lymph nodes. These cells are carefully washed twice, counted and finally re-suspended at ad hoc dilution in a ad hoc buffer (see below) washed.

### Immunological Read Out

Tetramer

Isolation of PBLs and tetramer staining method is the following: blood was taken from retro-orbital vein (50 μl per mouse, 10 mice per group) and directly diluted in RPMI+heparin (LEO) medium. PBLs were isolated through a lymphoprep gradient (CEDERLANE). Cells were then washed, counted and finally 1-5 10⁵ cells were resuspended in 50 μl FACS buffer (PBS, FCS1%, 0.002%NaN3) containing CD16/CD32 antibody (BD Biosciences) at 1/50 final concentration (f.c.). After 10 min., 50 μl of the tetramer mix was added to cell suspension. The tetramer mix contains 1 μl of siinfekl-H2Kb tetramer-PE from Immunomics Coulter. Anti-CD8a-PercP ( 1/100 f.c.) and anti-CD4-APC ( 1/200 F.c.) (BD Biosciences) antibodies were also added in the test. The cells were then left for 10 minutes at 37° C. before being washed once and analysed using a FACS Calibur™ with CELLQuest™ soft. 3000 events within the gate of living CD8 are acquired per test.

Intracellular Cytokine Staining (ICS)

ICS was performed on blood samples taken as described above. This assay include two steps: ex vivo stimulation and staining. Ex-vivo lymphocyte stimulation is performed in complete medium which is RPMI 1640 (Biowitaker) supplemented with 5% FCS (Harlan, Holland), 1 μg/ml (mix: 1/500) of each anti-mouse antibodies CD49d and CD28 (BD, Biosciences), 2 mM L-glutamine, 1 mM sodium pyruvate, 10 μg/ml streptamycin sulfate, 10 units/ml penicillin G sodium (Gibco), 10 μg/ml streptamycin 50 μM B-mercaptoethanol and 100× diluted non-essential amino-acids, all these additives are from Gibco Life technologies. Peptide stimulations were always performed at 37° C., 5% CO2.

1. Ex Vivo Stimulation:

Ova model: 5 to 10 10⁵ PBLs were re-suspended in complete medium supplemented a pool of 17 15-mer ova peptides (encompassing 11 different MHC classI-restricted peptides and 6 MHC class II-restricted peptides—here named pool 17) present at a concentration of each 1 μg/ml (= 1/5000 in mix). After 2 hours, 1 μg/ml ( 1/50 in mix) Brefeldin-A (BD, Biosciences) was added for 16 hours and cells were collected after a total of 18 hours.

2. Staining:

Cells were washed once and then stained with anti-mouse antibodies all purchased at BD, Biosciences; all further steps were performed on ice. The cells were first incubated for 10 min. in 50 μl of CD16/32 solution ( 1/50 f.c., FACS buffer). 50 μl of T cell surface marker mix was added ( 1/100 f.c. CD8a perCp, 1/100 f.c. CD4 APC Cy7) and the cells were incubated for 20 min. before being washed. Cells were fixed & permeabilised in 200 μl of perm/fix solution (BD, Biosciences), washed once in perm/wash buffer (BD, Biosciences) before being stained at 4° C. with anti IFNg-APC ( 1/50), anti-TNFa-PE( 1/100) and anti IL2-FITC ( 1/50) either for 2 hours or overnight. Data were analysed using a FACS Calibur™ with CELLQuest™ software.

15000 events within the gate of living CD8 are acquired per test.

Cell Mediated Cytotoxic Activity Detected In Vivo (CMC In Vivo)

To assess siinfekl-specific cytotoxicity, immunized and control mice were injected with a mixture of pulsed and not pulsed targets. Target pulse is obviously different according to the antigenic model while target labeling is identical for all antigenic model.

Ova model: mixture of target consists of 2 differentially CFSE-labeled syngeneic splenocyte and lymphnode populations, loaded or not with 1ng/ml of siinfekl peptide a 8-mers peptide known to be the immuno-dominant class I restricted-epitope.

For the differential labeling, carboxyfluorescein succinimidyl ester (CFSE; Molecular Probes—Palmoski et al.; 2002, J. Immunol. 168, 4391-4398) was used at a concentration of 0.2 μM or 2.5 μM. Both types of targets were pooled at 1/1 ratio and re-suspended at a concentration of 10⁸ targets/ml. 200 μl of target mix were injected per mouse into the tail vein at a defined time point. Cytotoxicity was assessed by FACS^{R} analysis on either blood (jugular vein) or spleen taken from sacrificed animal 18H after target injection). The mean percentage lysis of siinfekl-loaded target cells was calculated relative to antigen-negative controls with the following formula:

$$lysis \% = 100 - ( \frac{corrected target ( + )}{ control target ( - )} \times 100 )$$

$$Corrected target += target + \times \frac{( preinj . - )}{( preinj . + )}$$

Pre-injected target cells=mix of peptide-pulsed targets (preinj.+) and non-pulsed (preinj.−) targets acquired by FACS before injection in vivo.

Corrected target (+)=number of peptide-pulsed targets acquired by FACS after injection in vivo, corrected in order to take into account the number of preinj+ cells in the preinjected mix (see above).

Ag Specific Antibody Titer (Pooled Sera or Individual Sera Analysis of Total IgG): ELISA.

Serological analysis was assessed 15 days after second injection. Mice (10 per group) were bled by retro-orbital puncture.

Plate that are used are 96 well-plates (NUNC, Immunosorbant plates), their coating is different according to the antigen model:

Anti-ova and anti-LTcys total IgG were measured by ELISA. 96 well-plates were coated with antigen overnight at 4° C. (50 μl per well of antigen solutions respectively ova 10 μg/ml and LTxB-cys 2 μg/ml in PBS).

The plates were then washed in wash buffer (PBS/0.1% Tween 20 (Merck)) and saturated with 100 μl or 200 μl of saturation buffer (PBS/0.1% Tween 20/1% BSA/10% FCS) for 1 hour at 37° C. After 3 further washes in the wash buffer, 50 μl or 100 μl (function of the model) of diluted mouse serum was added and incubated for 60 minutes at 37° C. After another three washes, the plates were incubated for another hour at 37° C. with biotinylated anti-mouse total IgG diluted 1000 times in saturation buffer. After saturation 96w plates were washed again as described above.
A. Revelation of Anti-Ova ELISA

The plates were then washed in wash buffer (PBS/0.1% Tween 20 (Merck)) and saturated with 100 μl or 200 μl of saturation buffer (PBS/0.1% Tween 20/1% BSA/10% FCS) for 1 hour at 37° C. After 3 further washes in the wash buffer, 50 μl (function of the model) of diluted mouse serum was added and incubated for 60 minutes at 37° C. After another three washes, the plates were incubated for another hour at 37° C. with biotinylated anti-mouse total IgG diluted 1000 times in saturation buffer. After saturation 96w plates were washed again as described above. A solution of streptavidin peroxydase (Amersham) diluted 1000 times in saturation buffer was added, 50 μl per well. The last wash was a 5 steps wash in wash buffer.

Finally, 50 μl of TMB (3,3′,5,5′-tetramethylbenzidine in an acidic buffer—concentration of H₂O₂ is 0.01%—BIORAD) per well was added and the plates were kept in the dark at room temperature for 10 minutes

To stop the reaction, 50 μl of H₂SO₄ 0.4N was added per well. The absorbance was read at a wavelength of 450/630 nm by an Elisa plate reader from BIORAD. Results were calculated using the softmax-pro software,

B. Revelation of Anti-LTxBcys ELISA

The plates were then washed in wash buffer (PBS/0.1% Tween 20 (Merck)) and saturated with 100μl or 200 μl of saturation buffer (PBS/0.1% Tween 20/1% BSA/10% FCS) for 1 hour at 37° C. After 3 further washes in the wash buffer, 100 μl of diluted mouse serum was added and incubated for 60 minutes at 37° C. After another three washes, the plates were incubated for another hour at 37° C. with biotinylated anti-mouse total IgG diluted 1000 times in saturation buffer. After saturation 96w plates were washed again as described above. A solution of streptavidin peroxydase (Amersham) diluted 2000 times in saturation buffer was added, 100 μl per well. The last wash was a 5 steps wash in wash buffer.

Finally, 100 μl OPDA (37.5 μl ml Citrate de Na—0.05% tween—pH4.5+15 mg OPDA +37.5 μl H2O2 added extempo) per well was added and the plate were kept in the dark at room temperature for 20 minutes.

To stop the reaction, 100 μl of H₂SO₄ 2N was added per well. The absorbance was read at a wavelength of 490/630 nm by an Elisa plate reader from BIORAD. Results were calculated using the softmax-pro software.

3. Results

The results described below show that using either LT-ova, LTcys-ova or LTsiinfekl, the efficiency of a non-live vector system at inducing CD8 responses can be improved by combining it with adjuvant system A, H or G.

Evaluation of the Response with Adjuvant System A

FIGS. 1 to 5 and 19 to 22 show the CD8+ response to Ova when conjugated to LT or LTcys with and without ASA adjuvant. The graphs show that an improved immune response against Ova is observed when conjugated to the LTB or the LTBcys vector in combination with an adjuvant compared to the response seen with either unadjuvanted vectorized ovalbumin or non-vectorised ovalbumin administered with Adjuvant system A. This improved response is observed 6 days after the first injection for LT-ova (FIGS. 1 and 19), 14 days after the first injection for LTcys-ova (FIGS. 2 and 20) and up to day 88 after the second injection (FIGS. 3-5 and 21-22).

FIGS. 6 to 9 and 23-26 show that antigen-specific cytokine-producing CD8+ T-cells frequencies are increased when ovalbumin is conjugated to the LTB or the LTBcys vector and injected in combination with adjuvant ASA as compared to the response induced by conjugated ovalbumin alone or non-vectorised ovalbumin administered with Adjuvant system A. This improvement is most marked 6 to 60 days after the second injection (FIGS. 7 and 8).

FIGS. 10-13 and 27-30 illustrate improved CD4+ T-cells when vectorized antigen is used in combination with ASA compared to non-vectorized or non-adjuvanted compositions. The most improved percentage of Ova specific CD4+ T-cells is seen 14 days post after the first injection (FIGS. 10 and 28) and 6 days after the second injection (FIGS. 11 and 29).

FIGS. 14, 31 and 32 show that the Siinfekl-specific cytotoxic activity detected in vivo is improved when the antigen is both vectorized and administered with an adjuvant as tested either 21 days after the first injection (FIG. 31) or 12 (FIG. 14) or 65 (FIG. 32) days after the second injection.

FIGS. 15, 17 and 33 show the humoral response to Ova 14 days after the second injection. Antibody response against ova detected only when the vectorized antigen is adjuvanted. No improved antibody response is observed against ova when the antigen is conjugated to LT or LTcys and administered with ASA as compared to adjuvanted unconjugated ovalbumin,

FIGS. 16, 18 and 34 show that an anti-LT antibody response is raised by the adjuvanted vectorized antigen.

### Evaluation of the Response to Galactose-Purified LTcys-Ova

Conjugation products are usually heterogenous and may for instance contain variable amounts of LT conjugates that have lost the ability to bind to the GM1 receptor. FIGS. 19-26 show that LTcys-ova conjugates purified by molecular filtration can be further purified by galactose affinity and still show an improved CD8 response compared to unconjugated adjuvanted ovalbumin, which is better seen at 14 days post first injection (FIG. 20).

In addition, FIGS. 31 and 32 show that such a purified conjugate induces an improved Siinfekl-specific cytotoxic activity detected in vivo when administered with an adjuvant as tested either 21 days after the first injection (FIG. 31) or 65 days (FIG. 32) after the second injection.

FIG. 33 show that no improved antibody response is observed against ova when the antigen is conjugated to LT or LTcys and administered with ASA as compared to adjuvanted unconjugated ovalbumin,

FIG. 34 shows that an anti-LT antibody response is raised when the vectorized antigen is adjuvanted.

Evaluation of the Response with Adjuvant System H and G

FIGS. 35 and 36 compare StxB-ova and LT-ova with and without adjuvant systems A, H and G. The data clearly show that the response to antigen conjugated to either StxB or LT-B is increased when administered with these three adjuvant systems (FIG. 35) and at different doses with ASH (FIG. 36). The data demonstrate, together with the results from above, that the effect of including either adjuvant system increases the response to LT-ova similarly to StxB-Ova. It is therefore possible, with reference to Patent application WO2005/112991, to extrapolate that addition of the adjuvants listed in WO2005/112991 will show in improved immune response with LTB conjugated antigens similarly to that seen with StxB.

Evaluation of Response Using Alternative Non-Live Vectors with Adjuvant System A

The results (methods carried out as in 1.1-3 above) show that, measured at 7 days after a first injection, an increased CD8 response is better seen with LT-siinfekl adjuvanted with AS than is seen with adjuvanted non-vectorised Siinfekl or LT-siinfekl alone (FIG. 37). This improvement is seen at doses as low as 0.1 μg of LT-siinfekl. The same is seen with LF-siinfekl (FIG. 40), and with ExoA-siinfekl (FIG. 42). However, this improvement is not seen when measured 15 days post 1^{st} dose (FIGS. 38 and 41).

When looked at 7 days following a second injection, an improvement is again seen with LT-siinfekl (FIG. 39), LF-siinfekl and ExoA-siinfekl (FIG. 42) when combined with adjuvant system A, compared to either non-vectorised Siinfekl adjuvant or Siinfekl LFn or LT or ExoA without adjuvant.

What is claimed is:

1. A vaccine composition comprising the B-subunit of E. coli heat labile toxin or a derivative thereof with equal or greater than 90% homology complexed with an antigen and an adjuvant.

2. A vaccine composition as claimed in claim 1 wherein the B-subunit of E. coli heat labile toxin or a derivative thereof with equal or greater than 90% homology that binds the G_{M1} receptor.

3. A vaccine composition as claimed in claim 1 wherein the adjuvant is selected from the group of metal salts, oil in water emulsions, Toll like receptor ligands, saponins or combinations thereof.

4. A vaccine composition as claimed in claim 3 wherein the adjuvant is a Toll like receptor ligand.

5. A vaccine composition as claimed in claim 4 wherein the Toll like receptor ligand is an agonist.

6. A vaccine composition as claimed in claim 1, wherein the antigen and B-subunit of E. coli heat labile toxin or a derivative thereof with equal or greater than 90% homology are covalently attached.

7. A vaccine composition as claimed in claim 1 wherein the antigen and B-subunit of E. coli heat labile toxin or a derivative thereof with equal or greater than 90% homology are joined as a fusion protein.

8. A vaccine composition as claimed in claim 1 wherein the adjuvant is selected from the group: metallic salts, a saponin, lipid A or derivative thereof, an aminoalkyl glucosaminide phosphate, an immunostimulatory oligonucleotide or combinations thereof.

9. A vaccine composition as claimed in claim 8 wherein the saponin is presented as an Iscom, in an oil in water emulsion or in association with a liposome.

10. A vaccine composition as claimed in claim 8 wherein the saponin is QS21.

11. A vaccine composition as claimed in claim 8 wherein the Lipid A derivative is selected from Monophosphoryl lipid A, 3 deacylated Monophosphoryl lipid A, OM 174, OM 197, OM 294

12. A vaccine composition as claimed in claim 1 wherein the adjuvant is a combination of at least one representative from two of the following groups,
i) a saponin,
ii) a Toll—like receptor 4 ligand, and
iii) a Toll—Like receptor 9 ligand.

13. A vaccine composition as claimed in claim 12 wherein the saponin is QS21 and the toll like receptor 4 ligand is 3 deacylated monophosphoryl lipid A and the toll like receptor 9 is a CpG containing immunostimulatory oligonucleotide.

14. A vaccine composition as claimed in claim 1 wherein the antigen is selected from the group of antigens that provide immunity against the group of diseases selected from, intracellular pathogens or proliferative diseases.

15. A vaccine composition comprising a non-live vector derived from a bacterial toxin or an immunologically functional derivative thereof with an antigen and an adjuvant for use in medicine.

16. (canceled)

17. (canceled)

18. A method of treating or preventing disease comprising administering to a patient suffering from or susceptible to disease a vaccine composition according to claim 1.

19. A method for raising an antigen specific CD 8 immune response comprising the administration to a patient of a vaccine according to claim 1.

20. A process for the production of a vaccine according to claim 1 wherein an antigen complexed with the B-subunit of E. coli heat labile toxin or a derivative thereof with equal or greater than 90% homology that binds the G_{M1} receptor is admixed with an adjuvant.
